(19) United States
(12) Patent Application Publication
(10) Pub. No.: US 2005/0202447 A1
(43) Pub. Date: Sep. 15, 2005

(54) Array print buffers

(75) Inventors: Gary Opperman, St. Louis Park, MN (US); Asad Omar, Denver, CO (US)

Correspondence Address: MERCHANT & GOULD PC, P.O. BOX 2903, MINNEAPOLIS, MN, 55402-0903, US

(73) Assignee: SurModics, Inc., Eden Prairie, MN (US)

(21) Appl. No.: 10/800,043

(22) Filed: Mar. 11, 2004

(51) Int. Cl. C12Q 1/68; C12M 1/34; B05D 3/00

(52) U.S. Cl. 435/6; 435/287.2; 427/2.11

(57) ABSTRACT

The present invention relates to compositions suitable for spotting compounds onto a substrate, and methods employing these compositions. The composition can include one or more organic anions of formula I: R(X)ₘ(Y)ₙ. In an embodiment of formula I, R is an organic moiety, X is an anionic moiety, and, Y is a neutral hydrophilic moiety. The composition can include one or more neutral hydrophilic polymers. In an embodiment, the neutral hydrophilic polymer can be represented by formula V: 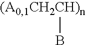 In Formula V, the group in parenthesis represents the polymer backbone, A can be absent or is a carbon or a heteroatom, and B is pendant neutral hydrophilic group. The present invention includes arrays, which can be made with or include compositions according to the present invention and can be made by using the method of the present invention.

### FIELD OF THE INVENTION

The present invention relates to compositions suitable for spotting compounds onto a substrate, and methods employing these compositions.

### BACKGROUND OF THE INVENTION

The immobilization of deoxyribonucleic acid (DNA) onto substrates has become an important aspect in the development of DNA-based assay systems including the development of microfabricated arrays for DNA analysis. Substrates for immobilization include the surface of microwell plates, tubes, beads, microscope slides, silicon wafers or membranes.

Hybridization is the method used most routinely to measure biomolecules, e.g., nucleic acids, by base pairing to probes immobilized on a solid support. When combined with amplification techniques such as the polymerase chain reaction (PCR) or ligase chain reaction (LCR), hybridization assays are a powerful tool for diagnosis and research.

A desirable goal for current DNA microarrays is the ability to put an entire species genome on one chip. Also, the ability to place replicates of a smaller set of genes on one chip is desirable. Another sought after goal is that the chips give results that represent the actual population of a specific nucleic acid in a sample. Although great strides have been made in this industry, problems remain. For example, certain surfaces or compounds being spotted can form uneven or irregular spots. Uneven or irregular spots can detract from the appearance and/or performance of a product.

Therefore, there remains a need for compositions that can more effectively spot compounds onto surfaces.

### SUMMARY OF THE INVENTION

The present invention relates to compositions suitable for spotting compounds onto a substrate, and methods employing these compositions.

In an embodiment, the present invention includes a composition. The composition can include one or more organic anions of formula I: R(X)ₘ(Y)ₙ. In an embodiment of formula I, R can include alkyl, alkenyl, alkynyl, and the like, or mixture thereof. In an embodiment of formula I, X is an anionic moiety. In an embodiment, each X can independently include carboxylate, phenol substituted with strongly electron withdrawing groups, phosphate, phosphonate, phosphinate, sulphate, sulphonate, thiocarboxylate, hydroxamate, and the like, or mixture thereof. In an embodiment of formula I, Y is a neutral hydrophilic moiety. In an embodiment, each Y can independently include amide, alcohol, ether, thiol, thioether, ester, thioester, borane, boric acid, metal complex, and the like, or mixture thereof. In an embodiment of formula I, m is about 1 to about 7. In an embodiment of formula I, n is 1 or more.

In an embodiment, the organic anion can include glucose-1-phosphate, glucose-6-phosphate, phytate, or mixture thereof.

In an embodiment, the present invention includes a composition. The composition can include one or more neutral hydrophilic polymers. In an embodiment, the neutral hydrophilic polymer can be represented by formula V: 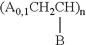
In Formula V, the group in parenthesis represents the polymer backbone, A can be absent or is a carbon (e.g., CH₂) or a heteroatom (e.g., O or N), n is about 100 to about 5000, and B is pendant neutral hydrophilic group (e.g., —OH, —OC(O)CH₃, —CONH₂, CONHR, —CONR₂, —OCH₃, —SH, —SCH₃, —COOR, —COSR, borane, boric acid, sulfone, amine oxide, and the like, or mixtures thereof). Examples of such neutral hydrophilic polymers of Formula V include polyvinyl alcohol, polyvinyl acetate, hydrolyzed polyvinyl alcohol, and the like, or mixtures thereof.

In an embodiment, the neutral hydrophilic polymer includes polyvinyl alcohol.

The composition can include buffer effective for maintaining pH of an aqueous form of the composition at greater than or equal to about 6 or greater than or equal to about 7.5. The composition can include compound suitable for being immobilized on a surface or support. The composition can include organic anion and/or neutral hydrophilic polymer in an amount effective to substantially decrease ring formation upon drying of a spot less than or equal to about 300 μm diameter, or about 10 to about 300 μm diameter, on a support.

In an embodiment, the present invention includes a method. The method can include a method of forming spots of a compound on a surface. The method can include a method of forming an array of spots of a compound on a surface. The method can employ compositions according to the present invention, which can include the present organic anion and/or neutral hydrophilic polymer. The method can employ compositions including one or more compounds suitable for being immobilized on the surface.

The present invention includes arrays. The arrays can be made with or include compositions according to the present invention. The arrays can be made by using the method of the present invention. In an embodiment, the array includes a plurality of spots on a solid support. One or more of the spots can include one or more compounds suitable for being immobilized on the surface.

### BRIEF DESCRIPTION OF THE FIGURES

FIG. 1 illustrates images of spots obtained by pin spotting with conventional print buffers and also with print buffers according to the present invention. The numbers above the sets of spots identify the oligos printed

FIGS. 2A through 2D schematically illustrate average appearance of centerline scans obtained for spots of the oligo BN30 printed with comparative print buffers (A, 50 mM sodium phosphate, and B, 50 mM sodium phosphate and 0.001% SDS) and for spots printed with print buffers according to the present invention (C, 200 mM glucose-6-phosphate, 50 mM sodium phosphate, and 0.001% SDS; and D, 25 mM phytate and 0.001% SDS). The illustrated lines are an average of 5 center lines per spot.

FIGS. 3A through 3D schematically illustrate average appearance of centerline scans obtained for spots of the oligo 5N009 printed with comparative print buffers (A, 50 mM sodium phosphate, and B, 50 mM sodium phosphate and 0.001% SDS) and for spots printed with print buffers according to the present invention (C, 200 mM glucose-6-phosphate, 50 mM sodium phosphate, and 0.001% SDS; and D, 25 mM phytate and 0.001% SDS). The illustrated lines are an average of 5 center lines per spot.

FIGS. 4A through 4D schematically illustrate average appearance of centerline scans obtained for spots of the oligo 5N021 printed with comparative print buffers (A, 50 mM sodium phosphate, and B, 50 mM sodium phosphate and 0.001% SDS) and for spots printed with print buffers according to the present invention (C, 200 mM glucose-6-phosphate, 50 mM sodium phosphate, and 0.001% SDS; and D, 25 mM phytate and 0.001% SDS). The illustrated lines are an average of 5 center lines per spot.

FIG. 5 illustrates images of spots obtained by pin spotting with comparative print buffers and also with print buffers according to the present invention.

FIGS. 6A through 6C schematically illustrate average appearance of centerline scans obtained for spots of the oligo RN103 printed with comparative print buffers (A, 50 mM sodium phosphate, and B, 50 mM sodium phosphate and 0.001% SDS) and for spots printed with print buffers according to the present invention (C, 1 mM sodium phosphate and 50 μg/ml polyvinyl alcohol). The illustrated lines are an average of 5 center lines per spot.

FIGS. 7A through 7C schematically illustrate average appearance of centerline scans obtained for spots of the oligo 5N009 printed with comparative print buffers (A, 50 mM sodium phosphate, and B, 50 mM sodium phosphate and 0.001% SDS) and for spots printed with print buffers according to the present invention (C, 1 mM sodium phosphate and 50 μg/ml polyvinyl alcohol). The illustrated lines are an average of 5 center lines per spot.

FIGS. 8A through 8E illustrate images of spots obtained with comparative print buffers and also with print buffers according to the present invention using piezoelectric printing.

### DETAILED DESCRIPTION OF THE INVENTION

Definitions

As used herein, the term “alkyl” refers to saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has 30 or fewer carbon atoms in its backbone (e.g., C₁-C₁₂ for straight chain, C₁-C₆ for branched chain). Likewise, cycloalkyls typically have from 3-10 carbon atoms in their ring structure, and preferably have 5, 6 or 7 carbons in the ring structure.

The term “alkyl” as used herein refers to both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an ester, a formyl, or a ketone), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocycle, an aryl alkyl, or an aromatic or heteroaromatic moiety. The moieties substituted on the hydrocarbon chain can themselves be substituted, if appropriate. For example, the substituents of a substituted alkyl can include substituted and unsubstituted forms of the groups listed above.

The phrase “aryl alkyl”, as used herein, refers to an alkyl group substituted with an aryl group (e.g., an aromatic or heteroaromatic group).

As used herein, the terms “alkenyl” and “alkynyl” refer to unsaturated aliphatic groups analogous in length and optional substitution to the alkyl groups described above, but that contain at least one double or triple bond respectively.

The term “aryl” as used herein includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with such substituents such as those described above for alkyl groups. The term “aryl” also includes polycyclic ring systems having two or more cyclic rings in which two or more carbons are common to two adjoining rings (the rings are “fused rings”) wherein at least one of the rings is aromatic, e.g., the other cyclic ring(s) can be cycloalkyls, cycloalkenyls, cycloalkynyls, aryls and/or heterocycles.

As used herein, the terms “heterocycle” or “heterocyclic group” refer to 3- to 12-membered ring structures, more preferably 3- to 7-membered rings, whose ring structures include one to four heteroatoms. Heterocycle groups include, for example, thiophene, thianthrene, furan, pyran, isobenzofuran, chromene, xanthene, phenoxanthin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents such as those described for alkyl groups.

As used herein, the term “heteroatom” as used herein means an atom of any element other than carbon or hydrogen, such as nitrogen, oxygen, sulfur and phosphorous.

As used herein, the term “about” modifying a quantity describing a feature the compositions or methods of the invention refers to variation in the numerical quantity that can occur, for example, through typical procedures used in making reagents for spotting and carrying out spotting procedures in the real world; through inadvertent error in these procedures; through differences in the manufacture, source, or purity of the ingredients or equipment employed to make the compositions or carry out the methods; and the like. Whether or not modified by the term “about”, the claims include equivalents to the quantities.

Compositions

The present invention relates to compositions suitable for spotting compounds onto a substrate. For example, one or more compounds can be suspended or dissolved in the composition of the invention and spotted onto a substrate. Advantageously, spotting with the present compositions can result in improved spot morphology.

The present compositions can supplement or replace conventional print buffers. For example, the present compositions include inventive buffer compositions containing the present additive (e.g., organic anion or neutral hydrophilic polymer). For example, the present compositions include inventive buffer compositions employing the present ingredient (e.g., organic anion) for buffering capacity. The present compositions can include a buffer effective to provide improved spot morphology on one or more array surfaces, compared to conventional print buffers. The present compositions can include a conventional print buffer plus an additive effective to provide improved spot morphology on one or more array surfaces, compared to the conventional print buffer lacking the present additive (e.g., organic anion or neutral hydrophilic polymer).

Compositions Including Organic Anion

The present compositions can include an organic anion. The organic anion can include alkyl, alkenyl, or alkynyl backbone; anionic moiety; and neutral hydrophilic moiety. Such an organic anion can be represented by Formula I: R(X)ₘ(Y)ₙ, in which R is the alkyl, alkenyl, or alkynyl backbone, X is the anionic moiety, Y is the neutral hydrophilic moiety, m is 1-7, and n is greater than 1.

The organic anion can include any of a variety of alkyl backbones. Suitable alkyl backbones include C1 to C12 linear, branched, or cyclic alkyl groups; linear, branched, or cyclic alkyl groups including substitution of a heteroatom for a carbon of the chain; saturated or unsaturated chains or rings; or the like. In an embodiment, the alkyl backbone includes a C4 to C8 cyclic system that can include one or more oxygen atoms in the ring. In an embodiment, the alkyl backbone includes a six member ring including carbon and, optionally, an oxygen atom. In an embodiment, the alkyl backbone with a six member ring also includes one or more carbons pendant to the ring. In an embodiment, the organic anion is not a surfactant.

The organic anion can include any of a variety of anionic moieties. Suitable anionic moieties (e.g., at neutral or basic pH in aqueous compositions) include carboxylates, phenols substituted with strongly electron withdrawing groups (e.g., substituted tetrachlorophenols), phosphates, phosphonates, phosphinates, sulphates, sulphonates, thiocarboxylates, hydroxamate, or the like. In an embodiment, the anionic moiety includes phosphate or sulfate. In an embodiment, the anionic moiety includes phosphate.

The organic anion can include any of a variety of neutral hydrophilic moieties. Suitable neutral hydrophilic moieties include amides, alcohols, ethers, thiols, thioethers, esters, thioesters, boranes, boric acids, metal complexes, and the like, or mixtures thereof. Suitable alcohols include primary alcohols, secondary alcohols, tertiary alcohols, aromatic alcohols, and the like.

In an embodiment, the organic anion has a structure represented by Formula II: 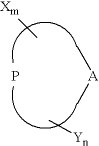
In Formula II, the alkyl backbone is cyclic, X is the anionic moiety, Y is the neutral hydrophilic moiety, A is carbon or heteroatom, m is 1-7, n is greater than 1, and P represents an additional 4-7 members of the cyclic backbone, e.g., carbon with up to one more heteroatom.

In an embodiment, the organic anion of Formula II has a structure represented by Formula III: 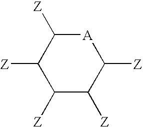
In Formula III, each Z is independently an anionic moiety (e.g., X) or a neutral hydrophilic moiety (e.g., Y), with at least one Z being an anionic moiety, and A is carbon or heteroatom. In an embodiment, Z is —OH, —OPO₃⁻, —CH₂—OPO₃⁻, or —OH (with at least one Z being —OPO₃⁻ or —CH₂—OPO₃⁻), and A is —CH—OPO₃⁻ or O. In an embodiment, Z is —OPO₃⁻ and A is CH—OPO₃⁻. In an embodiment, the organic anion of formula III is or includes glucose-1-phosphate, glucose-6-phosphate, phytate, or mixture thereof.

In an embodiment, the organic anion of Formula III has a structure represented by Formula IV: 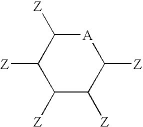
In Formula IV, each Z is independently an anionic moiety (e.g., X) or a neutral hydrophilic moiety (e.g., Y), with at least one Z being an anionic moiety, each Y is independently a neutral hydrophilic moiety, and A is a heteroatom. In an embodiment, Z is —OH, —OPO₃⁻ or —CH₂—OPO₃⁻ (with at least one Z being —OPO₃⁻ or —CH₂—OPO₃⁻), Y is —OH, and A is O. In an embodiment, the organic anion of formula IV is or includes glucose-1-phosphate, glucose-6-phosphate, or mixture thereof.

Although not limiting to the present invention, it is believed that the organic anion may interact with the substrate and the molecule being spotted in a way that impedes transport of the molecule being spotted to the perimeter of the drop or spot of fluid. This can be viewed as a process similar to chromatography. For example, in ion pair chromatography oppositely charged solutes can be used to change the chromatographic properties of an analyte. Ion pair chromatography of carboxylic acids employs a buffer containing triethylammonium ions. In this chromatography, the ammonium ions can complex the acid group, make the analyte more hydrophobic, and alter its chromatographic behavior.

Compositions Including Neutral Hydrophilic Polymer

The present compositions can include neutral hydrophilic polymer. The neutral hydrophilic polymer can include a polymer backbone and plurality of pendant neutral hydrophilic groups. Suitable neutral hydrophilic polymers include polyethers (e.g., polyethylene glycol or polypropylene glycol), substituted polyalkyleneimines (e.g., substituted polyethyleneimine), polyacrylamide, N- or N,N-substituted polyacrylamide, and the like. Suitable pendant neutral hydrophilic groups include, for example, amide, N-substituted amide, N,N-disubstituted amide, ester, ether, sulfone, amine oxide, alcohol, thiol, thioether, thioester, borane, boric acid, and the like. Suitable backbones for pendant neutral hydrophilic groups include, for example, alkyl, branched alkyl, polyether, and polyamine backbones, which can be formed from monomers such as vinyl monomers, acrylate ester monomers, secondary and tertiary acrylamide monomers, polyethylene glycol, polypropylene glycol, substituted polyethyleneimine, and the like. In an embodiment, the polymer backbone does not include carbohydrate moieties.

Suitable neutral hydrophilic polymers include vinylpyrrolidone, vinylcaprolactam, N-vinyl-N-methylacetamide, vinylmethylether, 2-vinylpyridine-N-oxide, vinylmethylsulfone, mixtures thereof, or the like; ethyleneglycol, ethyleneimine, PEG derivative of monomethacrylate (e.g., PEG 200, 400, or 1000), mixtures thereof, or the like; any of various aminimides or mixtures thereof; poly-(2-ethyloxazolene) (i.e. acetylated polyethyleneimine), polyvinylpyrrolidone (PVP), polyvinylcaprolactam, PVP-co-vinylacetate, polypropyleneglycolmonomethacrylate, mixtures thereof, or the like. Examples of the neutral hydrophilic polymers include polyvinyl alcohol, hydrolyzed polyvinyl alcohol, polyvinyl acetate, hydrolyzed polyvinyl acetate, mixtures thereof, or the like.

In an embodiment, the neutral hydrophilic polymer can include a vinyl backbone and pendant hydroxyl groups. Examples of such neutral hydrophilic polymers include polyvinyl alcohol.

In an embodiment, the neutral hydrophilic polymer has a general structure illustrated by Formula V: 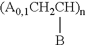
In Formula V, the group in parenthesis represents the polymer backbone, A can be absent or is a carbon (e.g., CH₂) or a heteroatom (e.g., O or N), n is about 100 to about 5000, and B is pendant neutral hydrophilic group (e.g., —OH, —OC(O)CH₃, —CONH₂, CONHR, —CONR₂, —OCH₃, —SH, —SCH₃, —COOR, —COSR, borane, boric acid, sulfone, amine oxide, and the like, or mixtures thereof). Examples of such neutral hydrophilic polymers of Formula V include polyvinyl alcohol, polyvinyl acetate, hydrolyzed polyvinyl alcohol, and the like, or mixtures thereof.

In an embodiment, the neutral hydrophilic polymer includes hydrolyzed polyvinyl alcohol. Hydrolyzed polyvinyl alcohol can be about 70 to about 100% hydrolyzed, about 80 to about 100% hydrolyzed, about 85 to about 100% hydrolyzed, or about 88% hydrolyzed.

In an embodiment of Formula V, A is absent, n is about 600 to about 1300, and B is —OH. Such neutral hydrophilic polymers of Formula V include polyvinyl alcohol. In an embodiment, the polyvinyl alcohol can be described as 88% hydrolyzed and/or having a molecular weight of 31-51 kD.

Although not limiting to the present invention, it is believed that the neutral hydrophilic polymer may interact with the substrate and the molecule being spotted in a way that impedes transport of the molecule being spotted to the perimeter of the drop or spot of fluid.

Buffer Compositions

The present invention includes buffer compositions of the present additives (e.g., organic anion or neutral hydrophilic polymer) with or without conventional buffer components.

In an embodiment, the present compositions include organic anion buffer compositions. Organic anion buffer compositions can include organic anion at pH within 1 or 2 pH units, or within 1 or 3 pH units, of the pKₐ of the organic anion and other conventional ingredients used in compositions for spotting molecules on substrates. Compositions employing the present organic anion as source of buffer capacity can also include anionic surfactant (e.g., sodium dodecyl sulfate (SDS), N-lauroylsarcosine, and the like, or mixture thereof), nonionic surfactant (e.g., polyoxyethylenesorbitan monolaurate, such as Tween 20), and the like, or mixtures thereof.

In an embodiment, the present organic anion buffer compositions can include an organic phosphate ester at pH about 6 to about 11, about 7.5 to about 9.5, about 8 to about 9, or about 8.5. In an embodiment, the organic phosphate ester can include glucose-1-phosphate, glucose-6-phosphate, phytate, and the like, or mixtures thereof. In an embodiment, the present organic anion buffer compositions can also include anionic surfactant (e.g., SDS). In an embodiment, the present organic anion buffer compositions have pH about 8.5.

In an embodiment, the present compositions include conventional buffers and the present organic anion and/or neutral hydrophilic polymer. The conventional buffers can include phosphate buffers, sulfate buffers, borate buffers, and the like, or mixtures thereof.

Suitable phosphate buffers include sodium phosphate, potassium phosphate, other phosphate salts, or mixtures thereof at pH of about 6 to about 11, about 7 to about 10, about 8 to about 9, or about 8.5. Suitable phosphate buffers include phosphate salt at concentration of about 10 to about 200 mM, about 30 to about 70 mM, or about 50 mM. Suitable phosphate buffers include 50 mM sodium phosphate at pH 8 to 8.5.

Additional Ingredients

The present compositions can include any of a variety of additional ingredients, particularly those employed in compositions for spotting a compound or compounds onto a polymer coated surface. The additional ingredients can include additional ingredients that can aid spotting and the compound to be spotted. Suitable additional ingredients include, for example, anionic surfactant, nucleic acid, nonionic surfactant, peptide, protein, carbohydrate, and the like, or mixtures thereof.

The present compositions can include anionic surfactant. Suitable anionic surfactants include sodium dodecyl sulfate, N-lauroylsarcosine, and the like, or mixture thereof.

The present print buffers can be employed for immobilizing any of a variety of biomolecules. The biomolecule can be unmodified or modified with a functional group to aid in immobilization. Suitable biomolecules include modified or unmodified oligo and polynucleotides (e.g., DNA or RNA), such as plasmid DNA, cosmid DNA, bacteriophage DNA, genomic DNA (including yeast, viral, bacterial, mammalian, insect, or like genomic DNA), cDNA, peptide nucleic acid, synthetic or natural RNA, tRNA, mRNA, O-methyl RNA, cloned DNA from artificial chromosomes (including HAC, BAC, MAC, PAC, YAC and like), and the like, or mixtures thereof. Suitable biomolecules include protein, carbohydrate, peptide, cell, tissue, and the like. In an embodiment, the biomolecule includes polypeptide or nucleic acid.

The biomolecule can optionally be functionalized or modified by any of a variety of known methods. For example, during synthesis, biomolecules, such as oligonucleotides or nucleic acids, can be prepared with functional groups such as amines or sulfhydryl groups in order to be reactive to NOS groups in the polymer composition. In an embodiment, the biomolecule includes a nucleic acid that includes an amine group, a sulfhydryl group, or a mixture thereof.

Methods Employing the Present Compositions

The present invention also includes methods employing compositions according to the present invention and including organic anion and/or neutral hydrophilic polymer. Such methods include methods of forming spots and/or arrays of a compound on a surface or support. In an embodiment, the present method includes a method of forming spots of a compound on a surface. Such a method can include applying to the surface a composition. The composition can include one or more compounds suitable for being immobilized on the surface and one or more of the organic anions according to the present invention. The composition can include one or more compounds suitable for being immobilized on the surface and one or more of the neutral hydrophilic polymers according to the present invention. The method can also include forming a spot on the surface.

The present method can spot any of a variety of compounds onto a surface. Compounds that can be immobilized on a surface include biomolecules such as a polypeptide (e.g., a receptor, an enzyme, or another protein), a nucleic acid, a peptide, a carbohydrate, or the like, or mixtures thereof.

In an embodiment, the present method forms a spot having advantageously desirable characteristics compared to spots of like size formed on the surface using conventional spotting compositions. For example, the present method can form a spot having distribution of compound characterized by a coefficient of variation of less than or equal to 50%, less than or equal to 40%, less than or equal to 30%, less than or equal to 25%, less than or equal to 15%, about 10% to about 30%, about 20% to about 30%, about 15% to about 25%, or about 10% to about 20%. In an embodiment, the composition includes organic anionic and/or neutral hydrophilic polymer in an amount effective to substantially decrease ring formation upon drying of a spot on a polymer coated support. The spot can have diameter less than or equal to about 300 μm, or about 10 to about 300 μm.

The present method can apply the composition using any of a variety of methods or apparatus for applying or spotting compounds on a solid support. For example, applying can include pin spotting, piezoelectric spotting, or ink jet spotting.

Spot Morphology

The present compositions and methods can be effective to provide improved spot morphology on one or more array surfaces, compared to conventional print buffers. In an embodiment, the present organic anion and/or neutral hydrophilic polymer can cause the improved spot morphology. Improved spot morphology can be evidenced by any of a variety of changes in the form, appearance, consistency, or regularity of the spot. In an embodiment, improved spot morphology manifests itself in greater consistency or less variation throughout the spot in distribution of the compound spotted on the surface or in signal representing that compound.

For example, the present compositions and methods can produce spots that show less of the so-called “doughnut” effect. Doughnut spots have a large amount of spotted compound or signal from that compound around the perimeter of the spot (the doughnut) and diminished amount of or signal from spotted compound in the interior of the spot (the doughnut hole). Beneficial effects of the present compositions and methods can appear as a decrease in the amount of or signal from compound at the perimeter relative to the interior of the spot. Beneficial effects of the present compositions and methods can appear as an increase in the amount of or signal from compound in the interior of the spot relative to the perimeter. This can be measured as a decrease in the coefficient of variation in the amount of or signal from compound distributed in regions (e.g., pixel sized regions) through or scattered within the spot.

Similarly, the beneficial effects of the present compositions and methods can appear as increased uniformity in the amount of or signal from compound within the spot. This can be measured as a decrease in the coefficient of variation in the amount of or signal from compound distributed in regions (e.g., pixel sized regions) through or scattered within the spot.

In an embodiment, the composition can include organic anion and/or neutral hydrophilic polymer in an amount effective to substantially decrease ring formation upon drying of a spot less than or equal to about 300 μm diameter on a polymer coated support.

Arrays

The present invention also includes arrays, such as arrays made with or including compositions according to the present invention. The arrays can be made by using the method of the present invention. In an embodiment, an array of the present invention includes a plurality of spots on a solid support. One or more of the spots can include one or more compounds suitable for being immobilized on the surface and one or more of the organic anions according to the present invention. One or more of the spots can include one or more compounds suitable for being immobilized on the surface and one or more of the neutral hydrophilic polymers according to the present invention.

In an embodiment, the present array or method employs a surface, such as a glass surface, coated with a polymer or other substance suitable for immobilizing a compound. Suitable surfaces for the present arrays or methods include those supplied by SurModics, Corning, Telechem, Takara, and other manufacturers. Suitable surfaces can be coated with polymers, such as acrylamide polymers, which can include functional groups for immobilizing a compound.

The present print buffer can be employed to form an array immobilized on the surface of a polymer coated slide. In an embodiment, coupling of a biomolecule to the surface can take place at pH 7-9 in a humid environment following printing the DNA solution in the form of small spots.

The present print buffer can be employed with known manufacturing or processing protocols, reagents, or equipment. For example, the present print buffers can be employed with commercially available micro-spotting robots (e.g., as available from Apogent Discoveries, Hudson, N.H. or Perkin Elmer, Foster City, Calif.). A microarray made with the present print buffer can include at least about 100/cm² (and preferably at least about 2,500/cm²) distinct bound biomolecules (e.g., polynucleotides or polypeptides). Each distinct bound biomolecule can be disposed at a separate, defined position in the array and can be deposited in a volume, for example, in the range of about 0.01 nL to about 100 nL. For example, the slide can be configured to receive sample in an amount of twenty nanoliters or less.

The regions (e.g., discrete spots) within the array can be generally circular in shape and can be separated from one another, for example, by about their largest diameter. A plurality of bound biomolecules can be provided, such that each region includes a single, and preferably different, bound biomolecule. In an embodiment, the spots are generally circular in shape, have a diameter of about 20 microns to about 150 microns, and are separated from other spots in the array by center to center spacing of about 40 microns to about 200 microns.

The present invention provides a method and print buffer composition for covalent attachment of bio- or other molecules onto the surface of a substrate, such as slides formed of organosilane-pretreated glass, organosilane-pretreated silicon, silicon hydride, or plastic. Silane treatment of the substrate surface, before the polymer composition is applied, can follow any of the procedures well known in the art. In an embodiment, the method and print buffer composition can immobilize nucleic acid probes onto plastic materials such as microwell plates, e.g., for use in hybridization assays. In an embodiment, the method and print buffer composition are adapted for use with substantially flat or molded surfaces, such as those provided by organosilane-pretreated glass, organosilane-pretreated silicon, silicon hydride, or plastic (e.g., polymethylmethacrylate, polystyrene, polycarbonate, polyethylene, or polypropylene). The print buffer composition can then be used to covalently attach a probe molecule such as a biomolecule (e.g., a nucleic acid) which in turn can be used for specific binding reactions (e.g., to hybridize a nucleic acid to a biomolecule).

Substrates (e.g., slides, microtiter plates, microspheres, microbeads, and polymer membranes) can be prepared from a variety of materials, including but not limited to plastic materials such as crystalline thermoplastics (e.g., high and low density polyethylenes, polypropylenes, acetal resins, nylons, and thermoplastic polyesters), amorphous thermoplastics (e.g., polycarbonates, polystyrene, and poly(methyl methacrylates), and glass. In an embodiment, suitable plastic or glass materials can provide a desired combination of such properties as rigidity, surface uniformity, resistance to long term deformation, and resistance to thermal degradation.

The present invention may be better understood with reference to the following examples. These examples are intended to be representative of specific embodiments of the invention, and are not intended as limiting the scope of the invention.

### EXAMPLES

### Example 1

### Anionic Buffer Additives Improve Spot Morphology in Contact Printing of Microarray Slides

In this Example, several organic anions were demonstrated to improve spot morphology, for example, by reducing the so-called “doughnut” effect.

Experiment 1—Glucose-6-Phosphate and Phytate Improve Spot Morphology

Materials and Methods

Amersham Codelink™ activated slides were arrayed with the RN103 probe (a 30-mer oligo modified with 3′-amine and 5′-TAMRA) and with the BN30 probe (a 30-mer oligo modified with 3′-amine and 5′-biotin) for density measurements. The surface was also arrayed with expression oligos 5N009, 5N021, and 5N041 (5′-amine modified 30-mer oligos for different human genes). These oligos have the structures represented below:

RN103-C6-NH₂ linker-
CCTGCGCCAGTTGAATGCCAGTGAGATAGA-TAMRA dye
BN30-C6-NH₂ linker-
GTCTGAGTCGGAGCCAGGGCGGCCGCCAAC-biotin
5N009-C6-NH₂ linker-
CTGGTTTTCTGCTCCTTGGTCCTGGGTGTC
5N021-C6-NH₂ linker-
CTGTCCCCTTTAGAGATCCCACCTGTCAGA
5N041-C6-NH₂ linker-
TTCCTTCATCCCTCTTGTTTCCCAGGTTTT

The oligos were suspended in print buffer at a concentration of 20 μM and printed on the slides using BioRobotics split pins and a BioRobotics arrayer. The compositions of the print buffers used are described in the Table reporting the results. After printing, the slides were incubated over-night at 75% relative humidity. The surface was blocked with 0.1 M TRIS pH 9.0 containing 50 mM ethanolamine followed by 5×SSC (0.75M NaCl and 0.075M sodium citrate) containing 0.1% N-lauroylsarcosine.

The slides were hybridized with biotinylated cRNA target prepared from human liver total RNA by in vitro transcription. Hybridization was detected using streptavidin Alexa 647. The slides were scanned using an Axon scanner (Union City, Calif.). The data was analyzed using GenePix software and the coefficient of variation (% CV) of the pixels within each spot was calculated.

Results

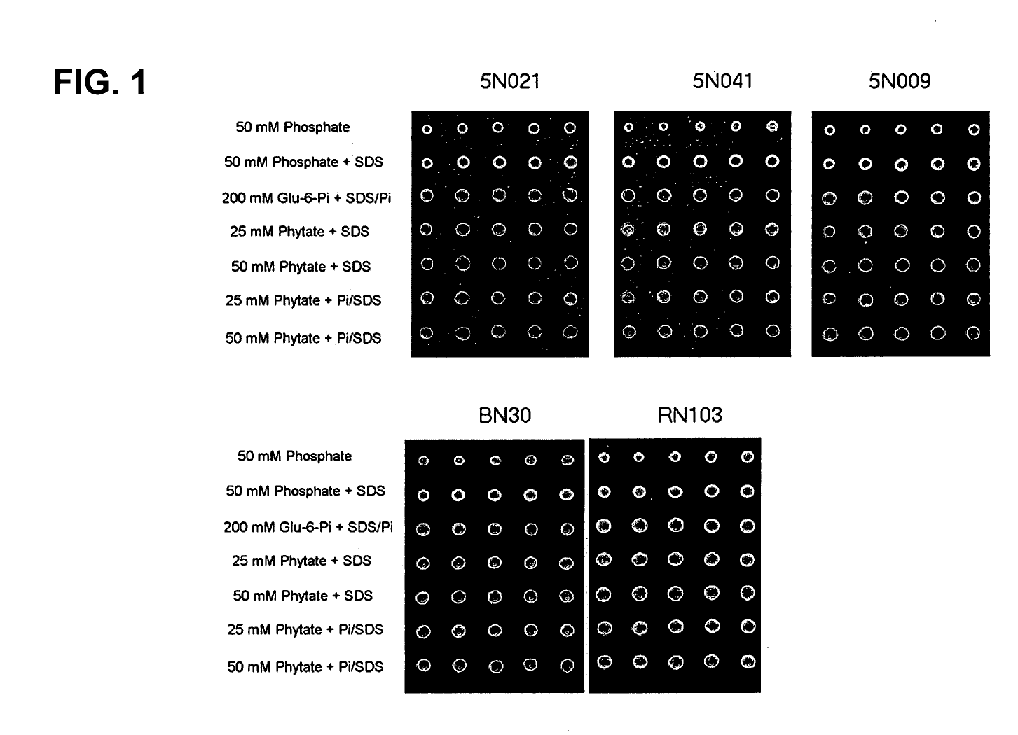

FIG. 1 illustrates spots obtained with the print buffers and additives described in Table 1. The images of the spots clearly show improved spot morphology with the organic anion additives to or replacements for the buffers. Table 1 reports the average % CV of spots printed with different print buffers. The results were obtained from four slides, each of which had been printed with 5 spots for each buffer and oligo.

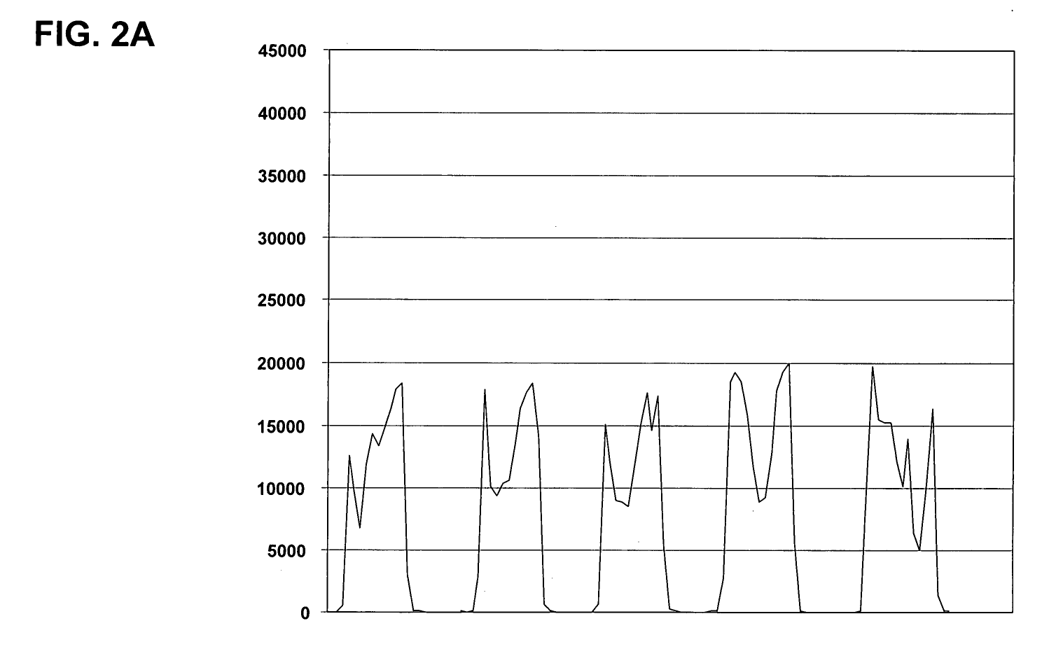

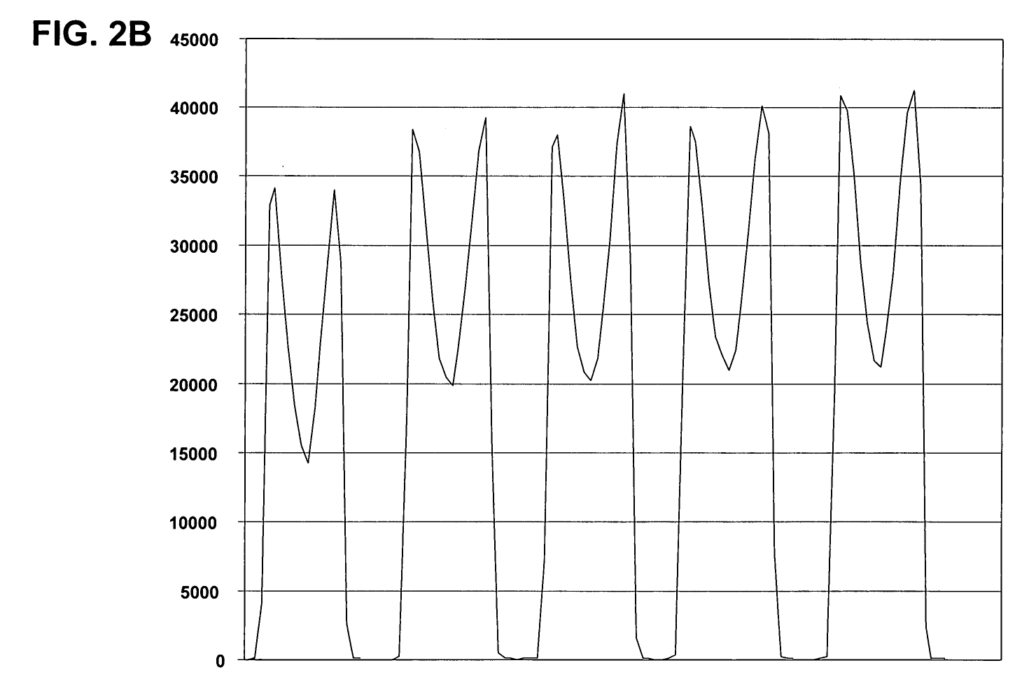

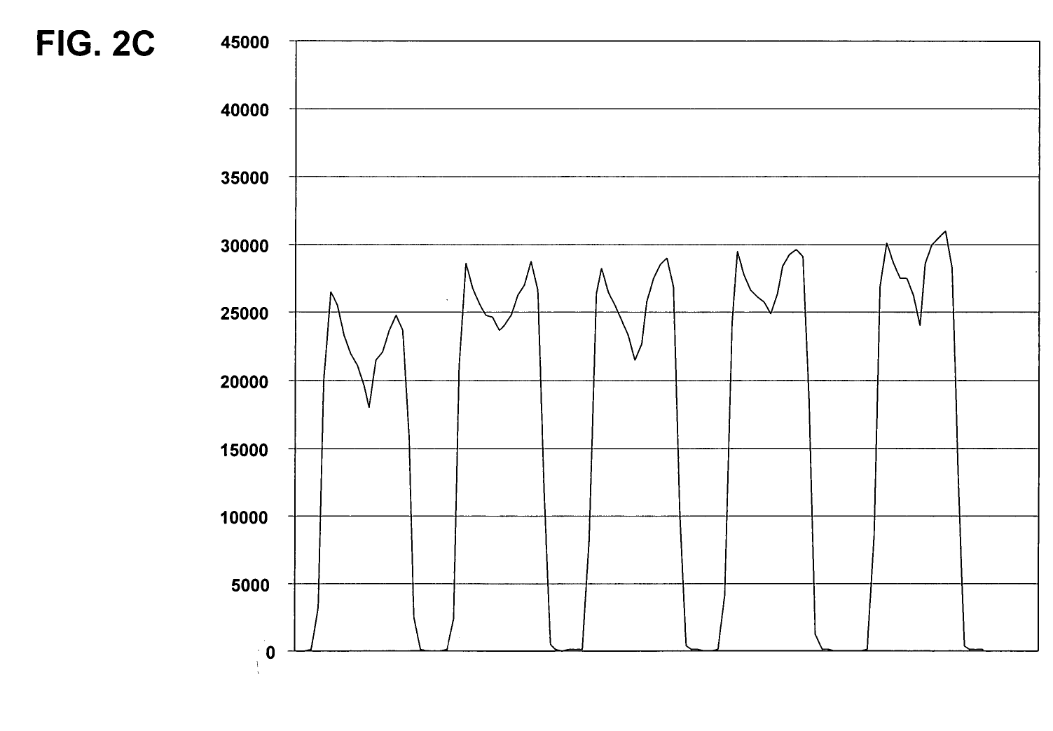

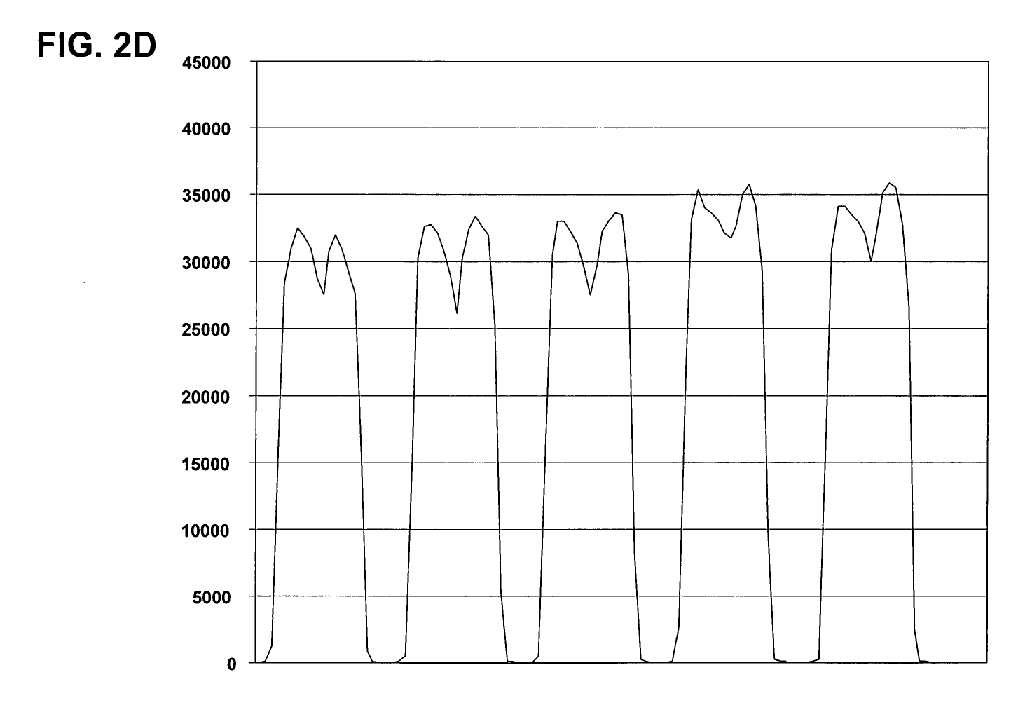

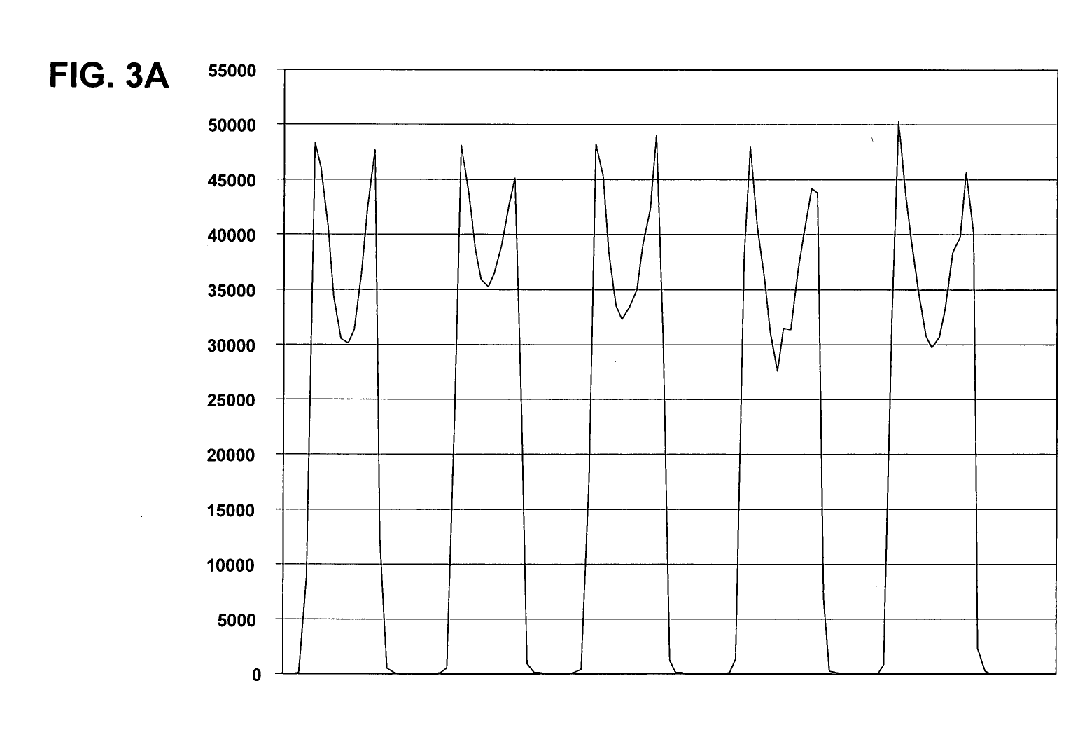

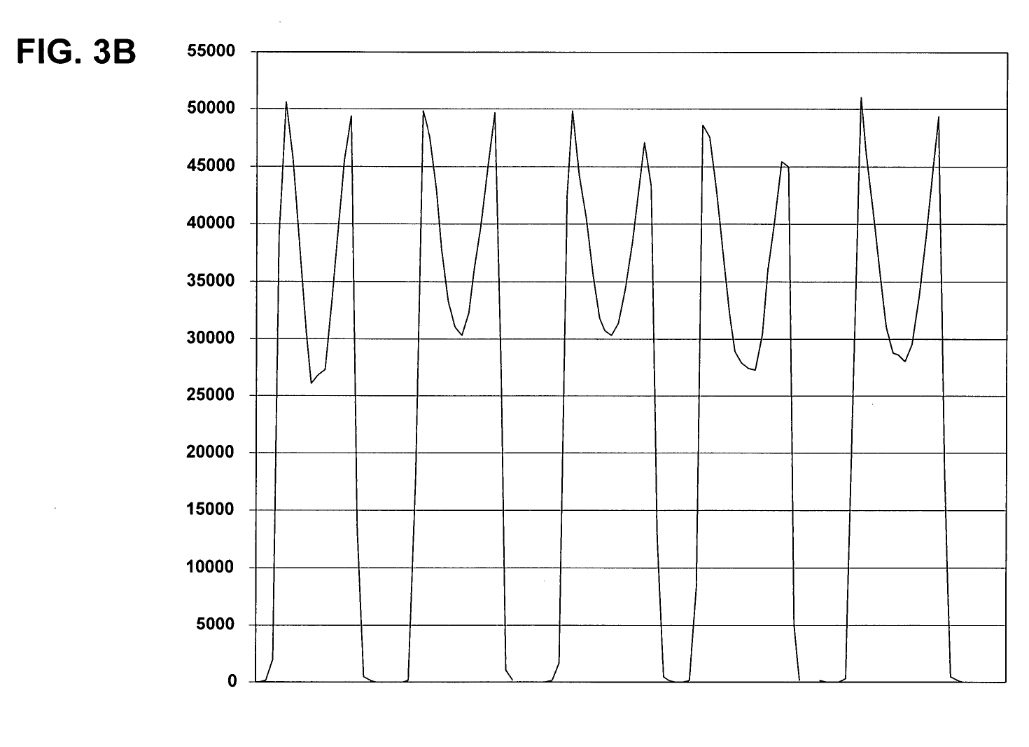

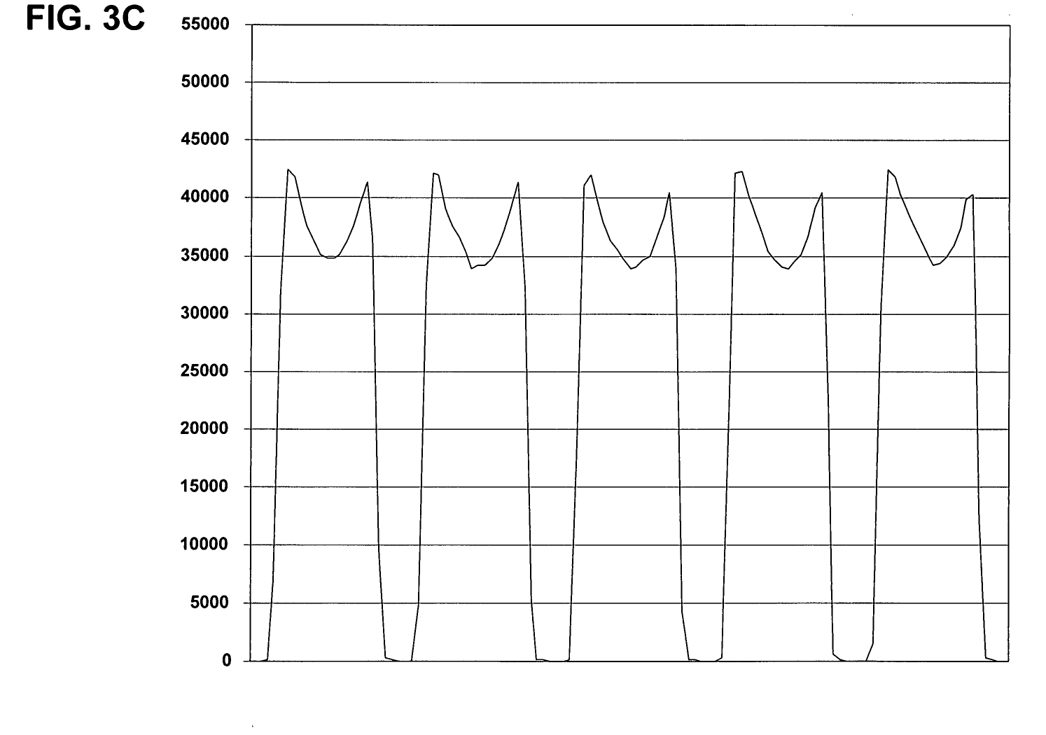

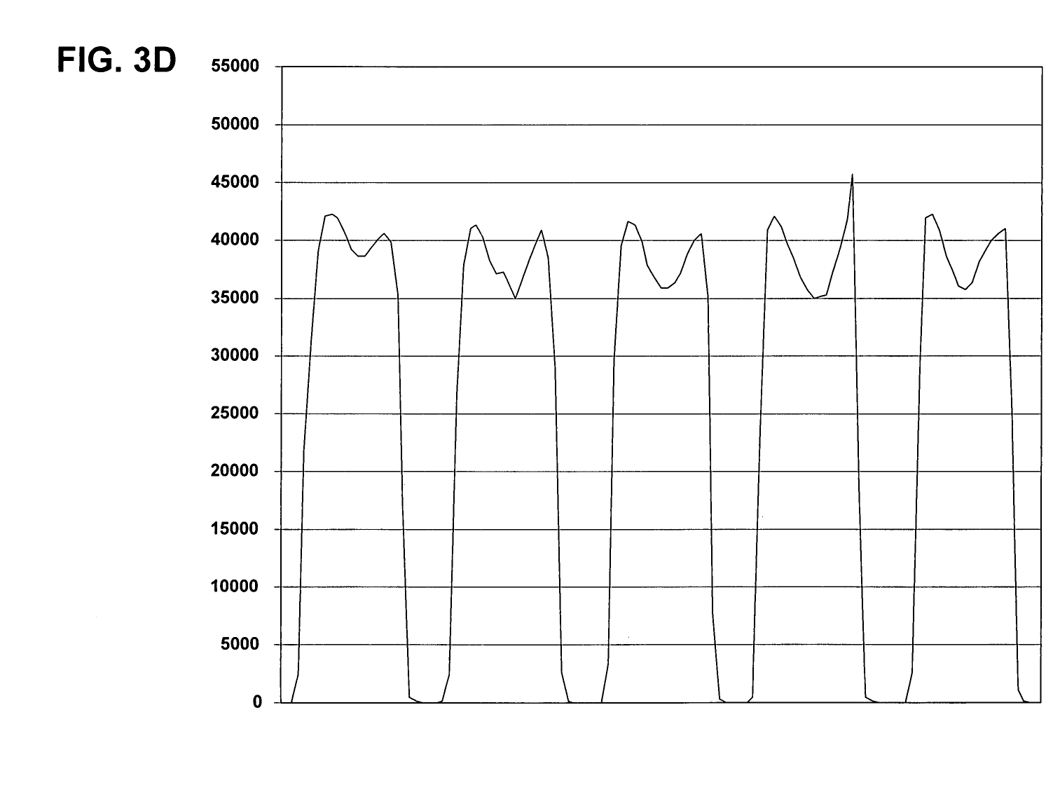

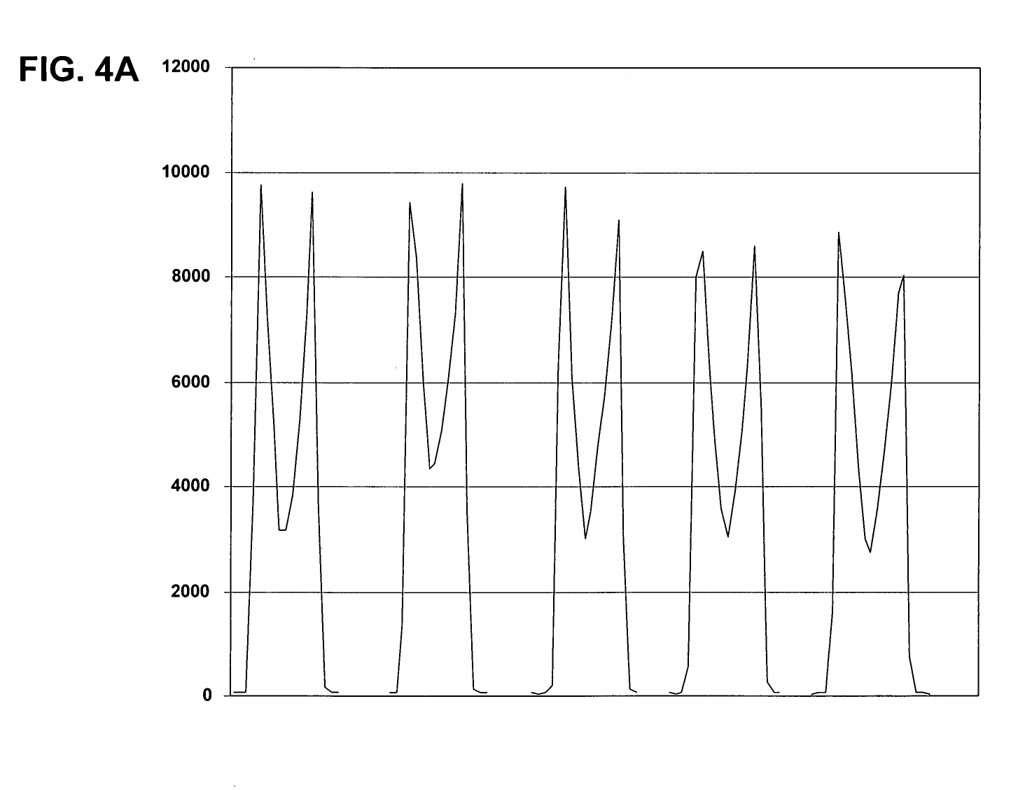

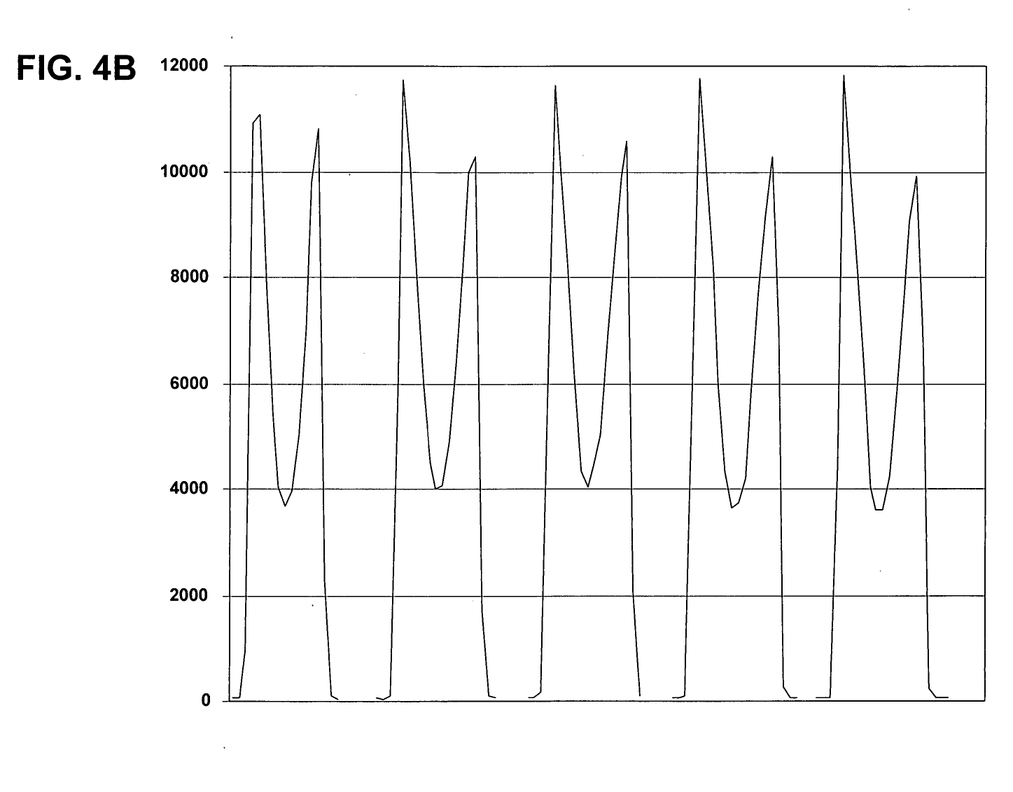

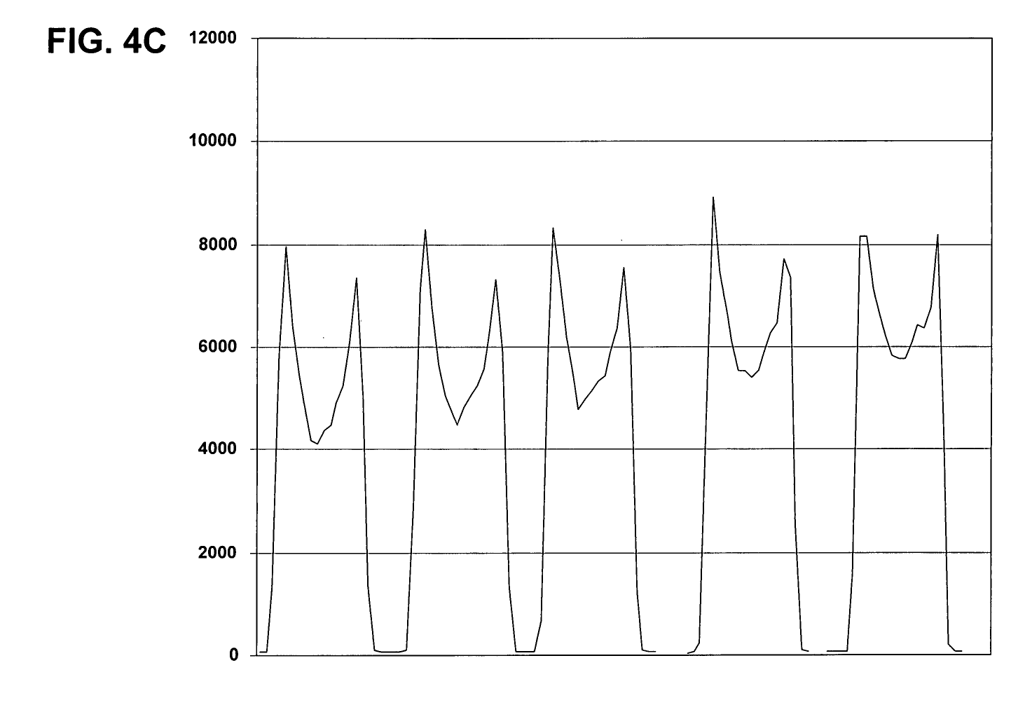

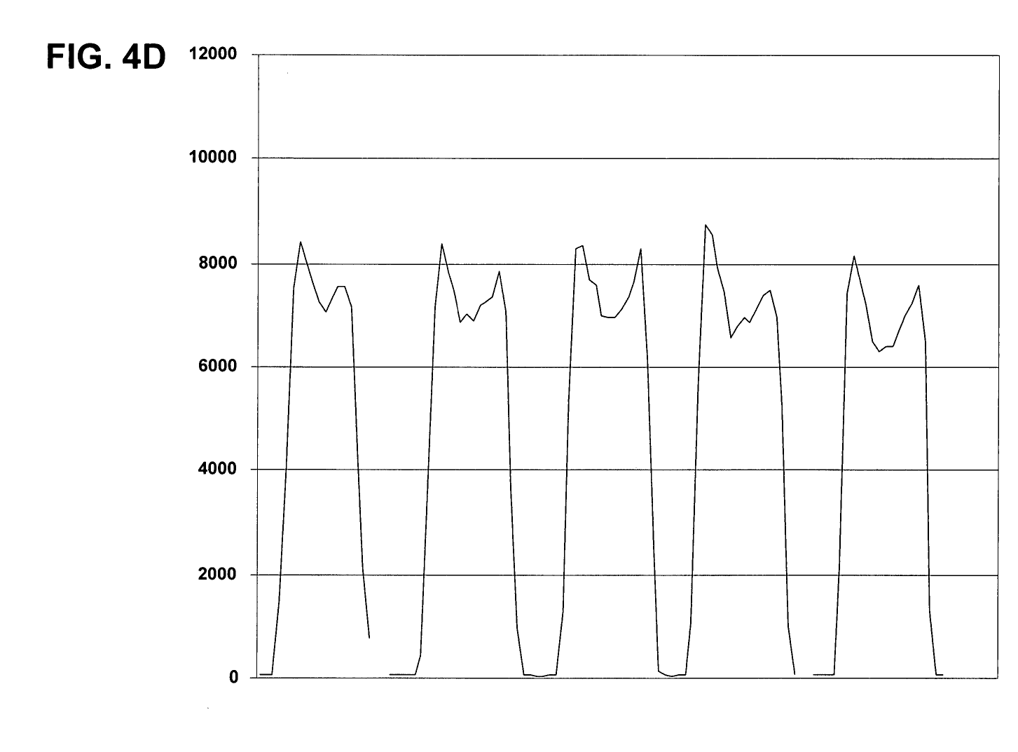

FIGS. 2A through 4D illustrate average appearance of centerline scans for spots with examples of the buffers/additives and spotted oligos. For each spotted oligo, the anionic additives to or replacements for the print buffers dramatically decrease the dip in signal (the doughnut hole) in the center of the spot.

TABLE 1
Coefficients of Variation (%) Produced by Spotting Various Oligos with Example and with Comparative
Compositions

| | | | | | | Overall |
|---|---|---|---|---|---|---|
| Print Buffer | 5N009 | 5N021 | 5N041 | BN30 | RN103 | Average |
| Comparative Buffers and Additives | | | | | | |
| 50 mM sodium phosphate, pH 8.5 | 23 | 47 | 40 | 39 | 32 | 36 |
| 50 mM sodium phosphate, pH 8.5 and 0.001% SDS | 26 | 38 | 34 | 25 | 32 | 31 |
| Example Buffers and Additives | | | | | | |
| 50 mM sodium phosphate, pH 8.5, 200 mM | 15 | 18 | 21 | 21 | 19 | 19 |
| glucose-6-phosphate, and 0.001% SDS | | | | | | |
| 50 mM sodium phosphate, pH 8.5, 25 mM sodium | 17 | 16 | 19 | 18 | 23 | 19 |
| phytate and 0.001% SDS | | | | | | |
| 50 mM sodium phosphate, pH 8.5, 50 mM sodium | 14 | 16 | 19 | 20 | 33 | 20 |
| phytate, and 0.001% SDS | | | | | | |
| 25 mM sodium phytate, pH 8.5, and 0.001% SDS | 19 | 15 | 22 | 19 | 24 | 20 |
| 50 mM sodium phytate, pH 8.5, and 0.001% SDS | 19 | 16 | 17 | 20 | 24 | 19 |

Conclusions

The organic anions glucose-6-phosphate and sodium phytate, both as additive to the print buffer and as the print buffer, significantly improved spot morphology as evidenced by reduced coefficient of variation of pixels throughout the spot.

Experiment 2—Glucose-1-Phosphate Improves Spot Morphology

Materials and Methods

The materials and methods and data acquisition and analysis were generally as described for Experiment 1, with the following exceptions. Amersham Codelink™ activated slides were arrayed with the RN118 probe (a 30-mer oligo modified with 3′-amine and 5′-TAMRA) and with the BN118 probe (a 30-mer oligo modified with 3′-amine and 5′-biotin) for density measurements. The surface was also arrayed with human gene oligos that gave signals in a hybridization assay—5N006, 5N009, 5N021, and 5N041 (5′-amine modified 30-mer oligos for different human genes). The RN118, BN118, and 5N006 oligo have the structures represented below:

RN118-C6-NH₂ linker-
GCCATGTGCAGTCTGGTTCAGGTTCATAAA-TAMRA dye
BN118-C6-NH₂ linker-
GCCATGTGCAGTCTGGTTCAGGTTCATAAA-biotin label
5N006-C6-NH₂ linker-TGATTGTGTTCTCTGCCTCTGGTTGACCTT

Results

Table 2 reports the average % CV of spots printed with different print buffers. The results were obtained from four slides, each of which had been printed with 20 spots for each buffer and oligo. The % CV data clearly shows improved spot morphology with the organic anion additives to the buffers. The addition of SDS only appears to have also enhanced the morphology in this experiment. The SDS addition by itself has been seen to sporadically give an improvement in spot morphology.

TABLE 2
Coefficients of Variation (%) Produced by Spotting Various Oligos with Example and with Comparative
Compositions

| | | | | | | | Overall |
|---|---|---|---|---|---|---|---|
| Print Buffer | 5N006 | 5N009 | 5N021 | 5N041 | BN118 | RN118 | Average |
| Comparative Buffers and Additives | | | | | | | |
| 50 mM sodium phosphate, pH 8.5 | 29 | 28 | 25 | 27 | 31 | 59 | 33 |
| 150 mM sodium phosphate, pH 8.5 | 23 | 29 | 23 | 24 | 22 | 73 | 32 |
| 50 mM sodium phosphate, pH 8.5 and 0.001% SDS | 19 | 21 | 17 | 19 | 21 | 26 | 20 |
| Example Buffers and Additives | | | | | | | |
| 50 mM sodium phosphate, pH 8.5, 200 mM | 18 | 12 | 17 | 16 | 18 | 26 | 18 |
| glucose-1-phosphate, and 0.001% SDS | | | | | | | |
| 50 mM sodium phosphate, pH 8.5, 200 mM | 14 | 24 | 15 | 20 | 20 | 29 | 20 |
| glucose-6-phosphate, and 0.001% SDS | | | | | | | |
| 50 mM sodium phosphate, pH 8.5, 25 mM sodium | 23 | 24 | 19 | 19 | 19 | 25 | 22 |
| phytate and 0.001% SDS | | | | | | | |

Conclusions

The organic anions glucose-1-phosphate, glucose-6-phosphate, and sodium phytate, significantly improved spot morphology as evidenced by reduced coefficient of variation of pixels throughout the spot.

Experiment 3—Anionic Print Buffers Improve the Spot Morphology on Other Commercially Available MicroArray Surfaces.

The materials and methods and data acquisition and analysis were as described for Experiment 2. The oligos were printed on Amersham Codelink™ and two other commercially available slides. These were amine oligo binding surfaces from Telechem (Sunnyvale, Calif.) and Takara (Otsu, Japan). Hybridization was done as described in Experiment 2.

Results

Table 3 reports the average % CV of spots printed with different print buffers. The combined average of all of the oligos in the experiment are reported. The results were obtained from two slides, each of which had been printed with 20 spots for each buffer and oligo. The % CV data clearly shows improved spot morphology with the organic anion additives to or replacements for the buffers.

TABLE 3
Coefficients of Variation (%) Produced by Spotting Various Oligos
with Example and with Comparative Compositions.
Numbers are averages for all of the oligos in the experiment.

| | Code-Link | Telechem | |
|---|---|---|---|
| | Activated | Epoxide | |
| Print Buffer | Slide | Slide | Takara |
| Comparative Buffers and Additives | | | |
| 50 mM sodium phosphate, pH 8.5 | 24 | 31 | 34 |
| 150 mM sodium phosphate, pH 8.5 | 30 | 34 | 33 |
| 50 mM sodium phosphate, pH 8.5, | 21 | 27 | 24 |
| and 0.001% SDS | | | |
| Example Buffers and Additives | | | |
| 50 mM sodium phosphate, pH 8.5, | 20 | 23 | 20 |
| 200 mM glucose-1-phosphate | | | |
| and 0.001% SDS | | | |
| 50 mM sodium phosphate, pH 8.5, | 23 | 28 | 19 |
| 200 mM glucose-6-phosphate | | | |
| and 0.001% SDS | | | |
| 50 mM sodium phosphate, pH 8.5 | 23 | 24 | 22 |
| and 25 mM sodium phytate | | | |
| and 0.001% SDS | | | |

Conclusions

The organic anions glucose-1-phosphate, glucose-6-phosphate, and sodium phytate, significantly improved spot morphology on the hydrophilic Codelink™ surface and more hydrophobic commercial amine binding surfaces.

### Example 2

### Neutral Hydrophilic Polymer Improves Spot Morphology

In this Example, a neutral hydrophilic polymer additive was demonstrated to improve spot morphology, for example, by reducing the so-called “doughnut” effect.

Materials and Methods

The materials and methods and data acquisition and analysis were generally as described for Example 1. The oligos included NC30 probe (a 30-mer oligo modified with 3′-amine and 5′-Cy3) for density measurements and 5N009 was used as an oligo that would give a signal in a hybridization experiment. The oligo NC30 has the structure represented below:

NC30-Cy3-GTCTGAGTCGGAGCCAGGGCGGCCGCCAAC-C6-NH₂
linker.

The content of the print buffers are shown in the table below. The print buffer included polyvinyl alcohol (PVA), including PVA that was 87-89% hydrolyzed.
Results

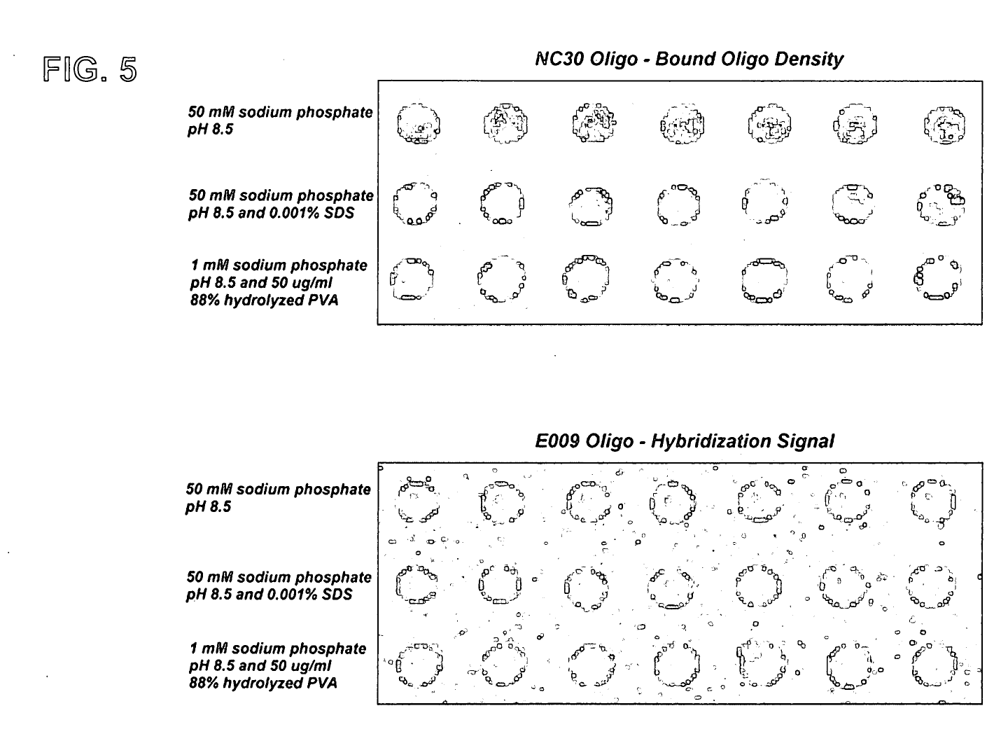

FIG. 5 illustrates spots obtained with the print buffers and additives described in Table 4. The number under each spot is the % CV obtained for that spot. Table 4 reports the average % CV of spots printed with different print buffers. The images of the spots and the % CV raw data clearly show improved spot morphology with the neutral hydrophilic polymer additive to the buffer. The results were obtained from four slides, each of which had been printed with 5 spots for each buffer and oligo.

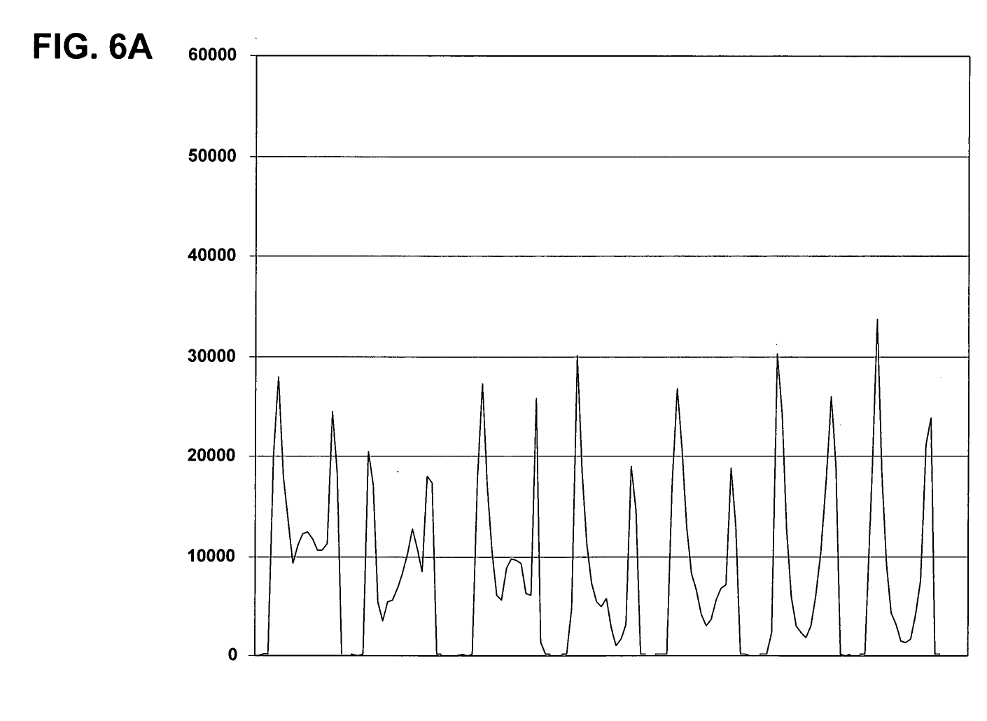

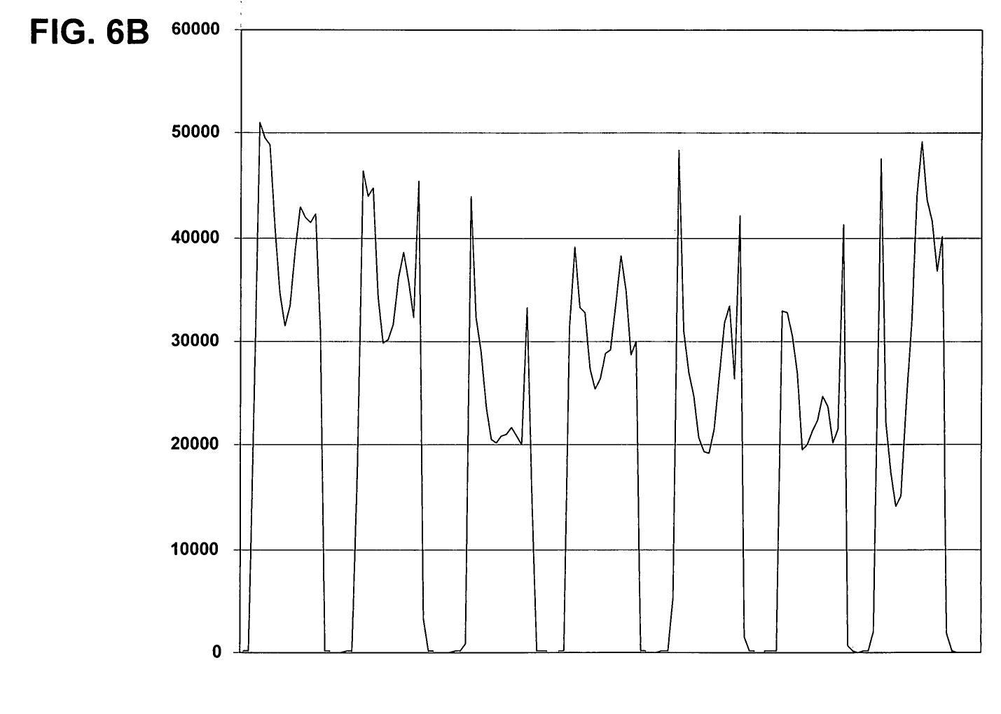

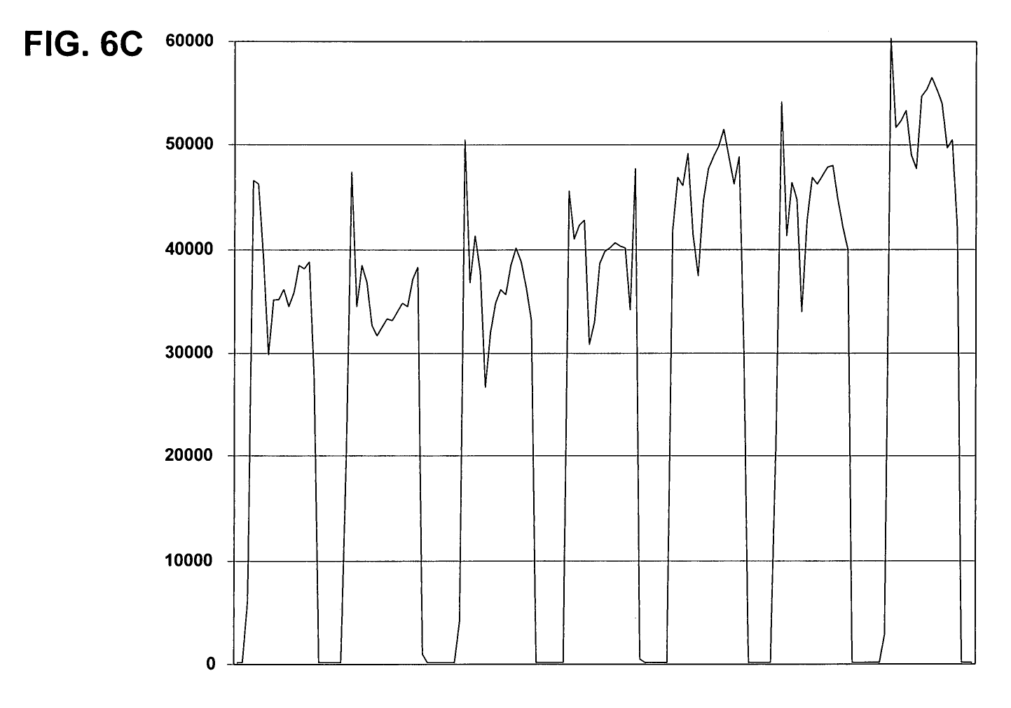

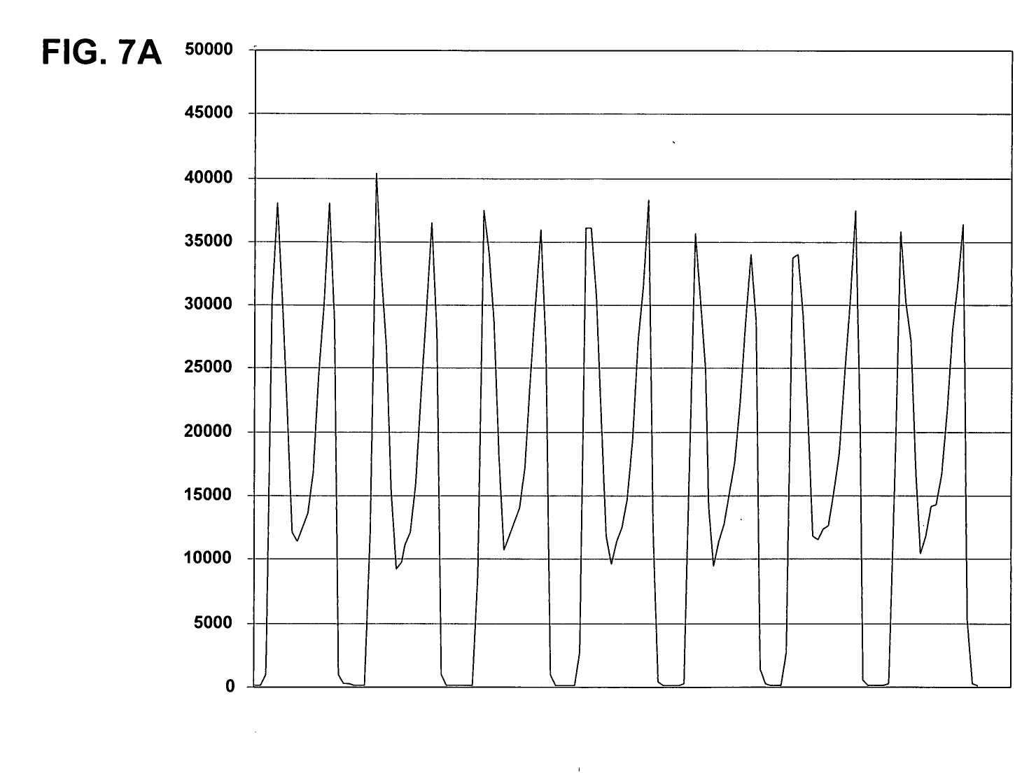

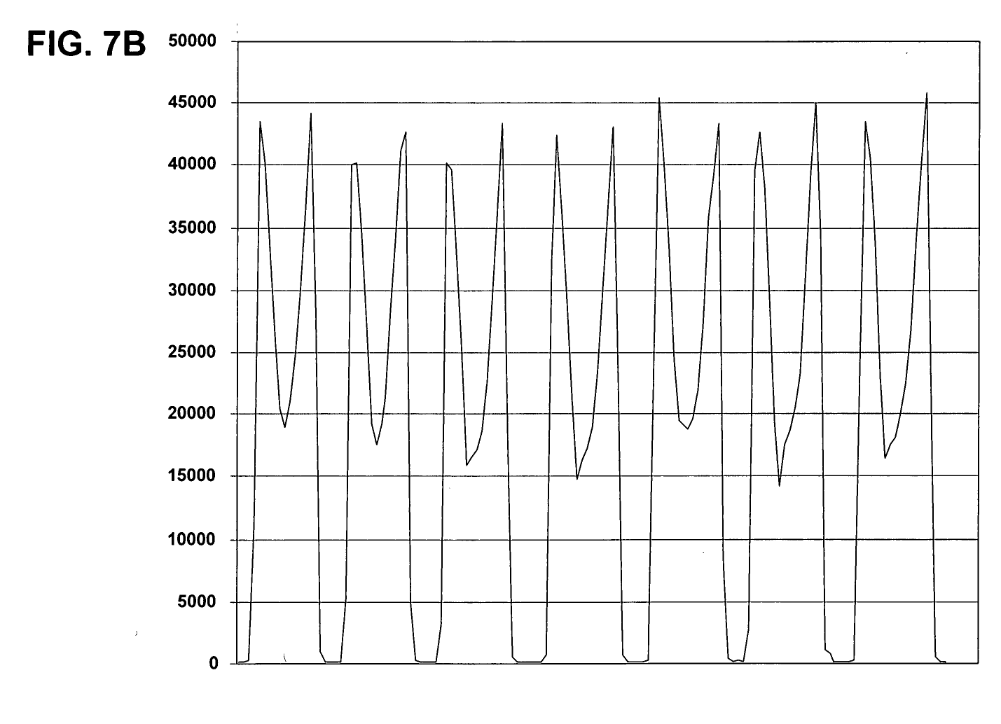

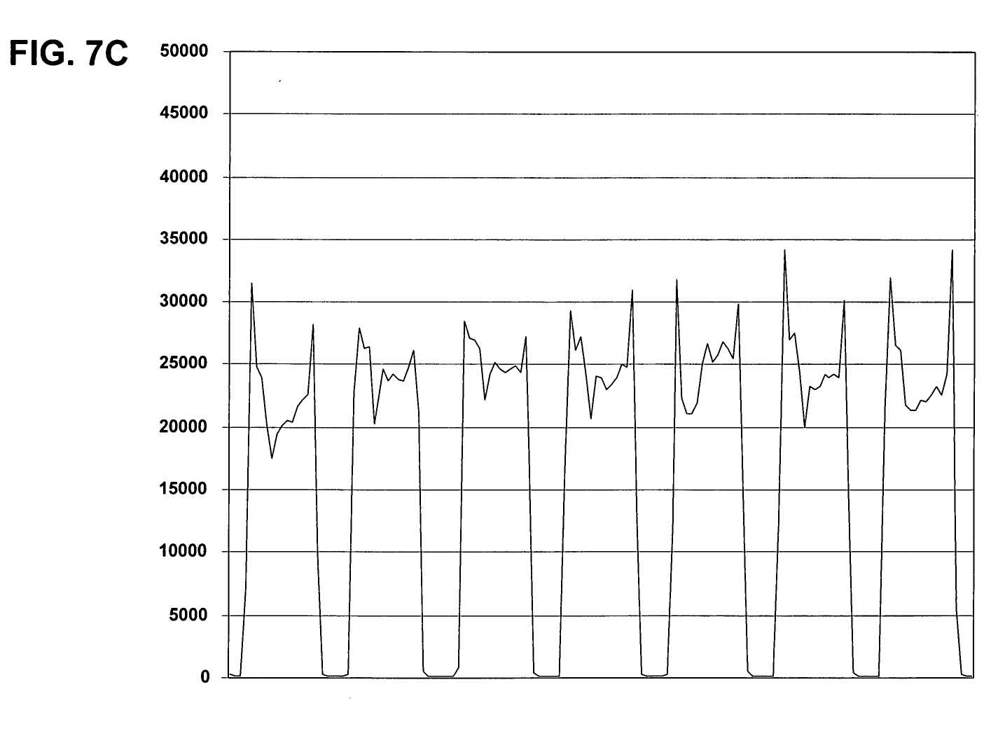

FIGS. 6A through 7C illustrate average appearance of centerline scans for spots with each of the buffers/additives and spotted oligos. For each spotted oligo, the print buffer formulation containing PVA dramatically decreased the dip in signal (the doughnut hole) in the center of the spot

TABLE 4
Coefficient of Variation (%) Produced by Spotting Various Oligos
with Example and with Comparative Compositions

| Print Buffer | NC30 | 5N009 |
|---|---|---|
| Comparative Buffers and Additives | | |
| 50 mM sodium phosphate, pH 8.5 | 73 | 49 |
| 50 mM sodium phosphate, pH 8.5, and 0.001% SDS | 34 | 40 |
| Example Buffer and Additive | | |
| 1 mM sodium phosphate, pH 8.5 with 50 μg/mL | 20 | 21 |
| polyvinyl alcohol (31-50K, 88% hydrolyzed) | | |

Conclusions

The neutral hydrophilic polymer polyvinyl alcohol significantly improved spot morphology as evidenced by reduced coefficient of variation of pixels throughout the spot.

### Example 3

### Formulations of the Present Invention Improve the Spot Morphology Produced by Piezoelectric Printing

Materials and Methods

The materials and methods and data acquisition and analysis were as described for Experiment 1. The exception was that the oligo solutions were printed using a piezoelectric robot.

Results

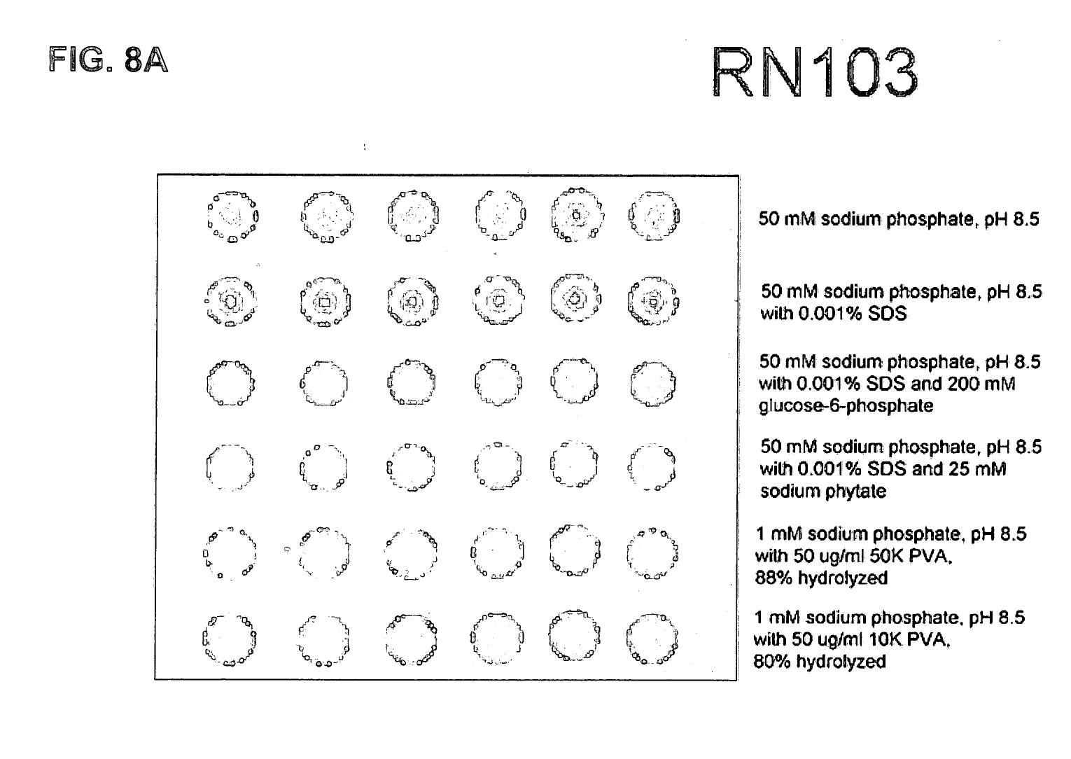

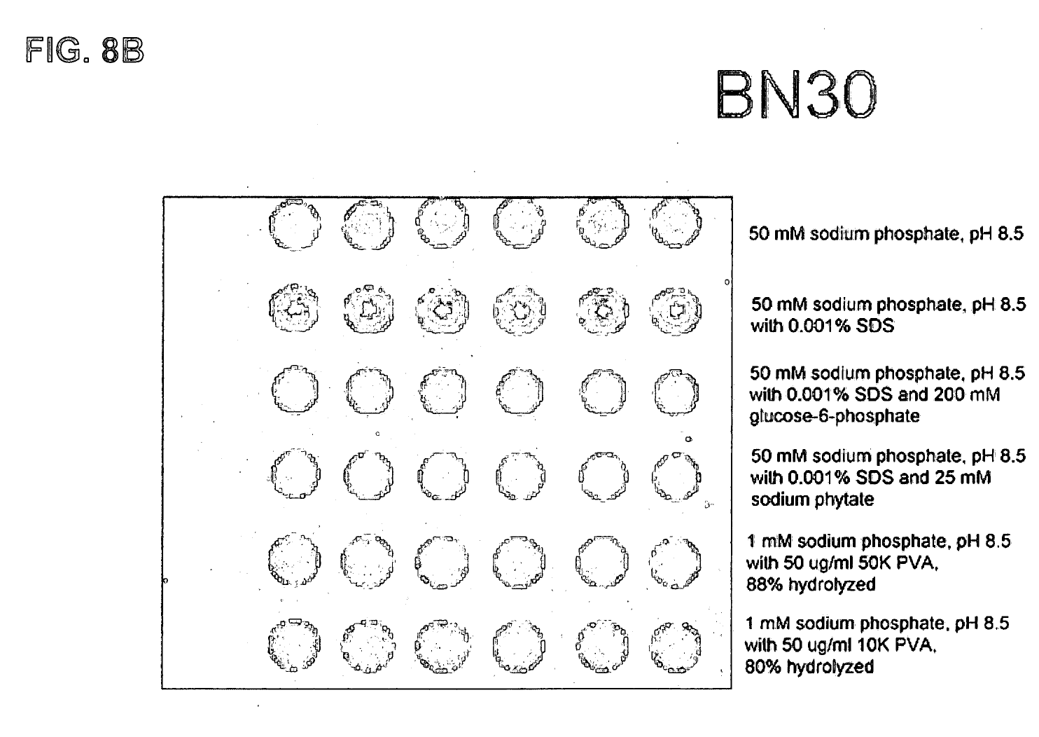

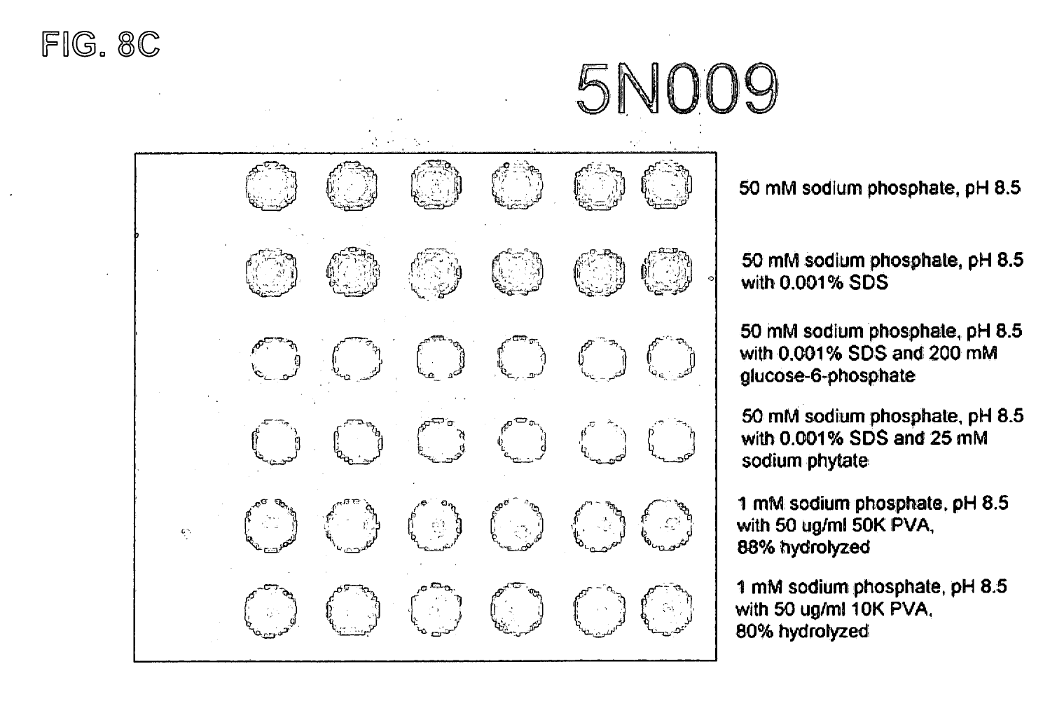

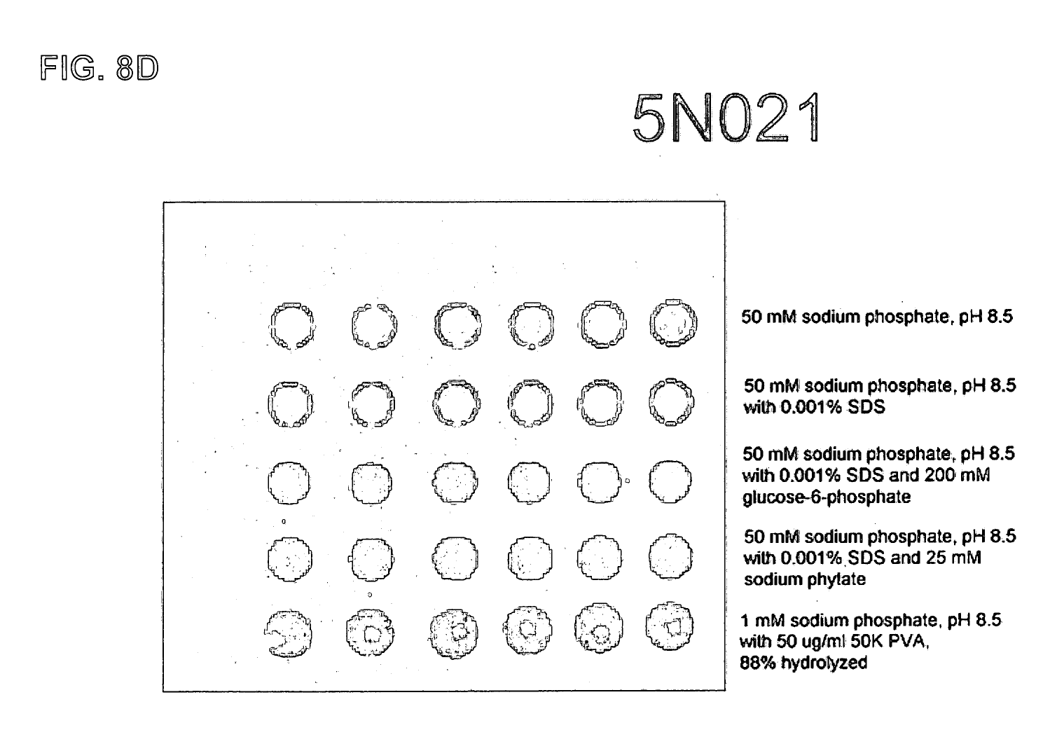

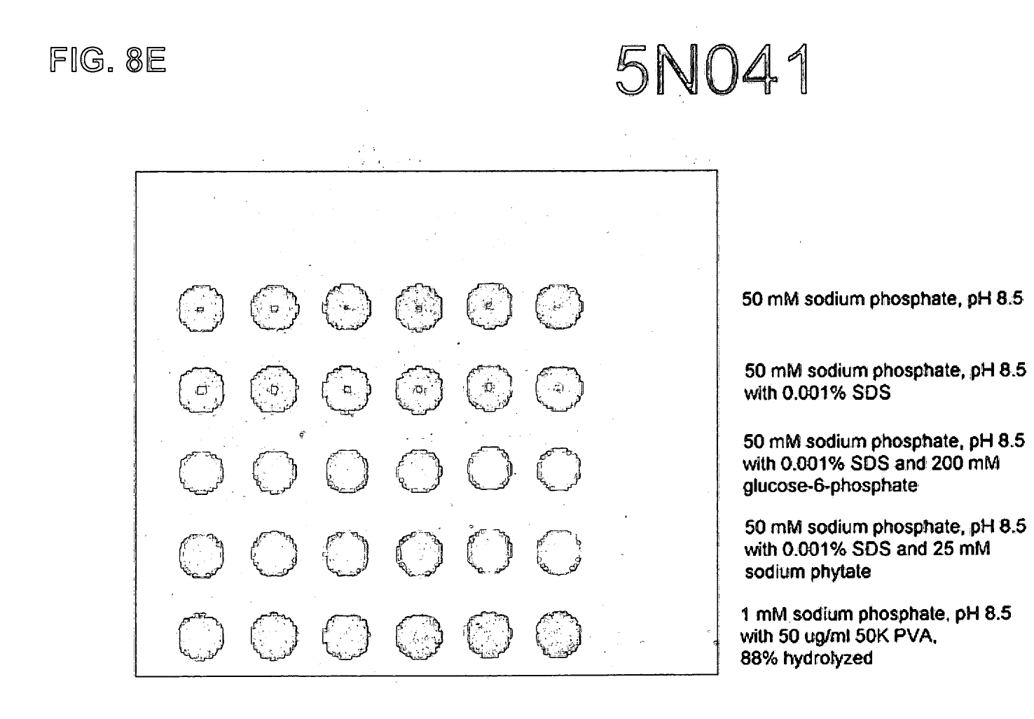

FIGS. 8A through 8E illustrate spots obtained by piezoelectric spotting with the print buffers and additives described in Table 5. Table 5 reports the average % CV of spots printed with different print buffers. The images of the spots and the % CV raw data clearly show improved spot morphology with the organic anion additives to or replacements for the buffers. The results were obtained from four slides, each of which had been printed with 40 spots for each buffer and oligo.

TABLE 5
Coefficient of Variation (%) Produced by Piezoelectric Spotting of Various Oligos with Example and
with Comparative Compositions

| | | | | | | Overall |
|---|---|---|---|---|---|---|
| Print Buffer | 5N009 | 5N021 | 5N041 | BN30 | RN103 | Average |
| Comparative Buffers and Additives | | | | | | |
| 50 mM sodium phosphate, pH 8.5 | 36 | 61 | 37 | 60 | 53 | 49 |
| 50 mM sodium phosphate, pH 8.5 and 0.001% SDS | 33 | 65 | 42 | 30 | 50 | 44 |
| Example Buffers and Additives | | | | | | |
| 50 mM sodium phosphate, pH 8.5, 200 mM | 13 | 14 | 13 | 18 | 19 | 15 |
| glucose-6-phosphate and 0.001% SDS | | | | | | |
| 50 mM sodium phosphate, pH 8.5, 25 mM sodium | 14 | 26 | 19 | 19 | 23 | 20 |
| phytate and 0.001% SDS | | | | | | |
| 1 mM sodium phosphate, pH 8.5 with 50 μg/mL | 22 | 32 | 32 | 21 | 23 | 26 |
| polyvinyl alcohol (31-50K, 88% hydrolyzed) | | | | | | |

Conclusions

All formulations of the present invention significantly improved spot morphology as evidenced by reduced coefficient of variation of pixels throughout the spot. This is true regardless of the method used for spotting (e.g., pin spotting and piezoelectric spotting).

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise. It should also be noted that, as used in this specification and the appended claims, the phrase “adapted and configured” describes a system, apparatus, or other structure that is constructed or configured to perform a particular task or adopt a particular configuration to. The phrase “adapted and configured” can be used interchangeably with other similar phrases such as arranged and configured, constructed and arranged, adapted, constructed, manufactured and arranged, and the like.

All publications and patent applications in this specification are indicative of the level of ordinary skill in the art to which this invention pertains.

The invention has been described with reference to various specific and preferred embodiments and techniques. However, it should be understood that many variations and modifications may be made while remaining within the spirit and scope of the invention.

1. A composition comprising:
a buffer effective for maintaining pH of aqueous composition at greater than or equal to about 6; and
an organic anion of formula I: R(X)ₘ(Y)ₙ; in which:
R is alkyl, alkenyl, or alkynyl;
each X is independently carboxylate, phenol substituted with strongly electron withdrawing groups, phosphate, phosphonate, phosphinate, sulphate, sulphonate, thiocarboxylate, hydroxamate, or combination thereof;
each Y is independently amide, alcohol, ether, thiol, thioether, ester, thioester, borane, boric acid, or metal complex;
m is 1-7; and
n is greater than 1;
the organic anion being effective to substantially decrease ring formation upon drying of a spot less than or equal to about 300 μm diameter on a support.

2. The composition of claim 1, wherein each X is independently phosphate or sulfate.

3. The composition of claim 1, wherein each Y is hydroxyl.

4. The composition of claim 1, wherein the organic anion of formula I has the structure of represented by formula II: 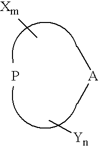
in which P represents an additional 4-7 members of cyclic backbone comprising carbon with up to one heteroatom.

5. The composition of claim 4, wherein each X is independently phosphate or sulfate.

6. The composition of claim 4, wherein each Y is hydroxyl.

7. The composition of claim 1, wherein the organic anion of formula I has the structure of represented by formula III: 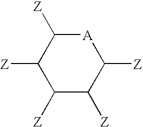
in which:
Z is —OH, —OPO₃⁻ or —CH₂—OPO₃⁻; and
at least one Z is —OPO₃⁻ or —CH₂—OPO₃⁻;
Y is —OH; and
A is —CH—OPO₃⁻ or O.

8. The composition of claim 7, wherein the organic anion is glucose-1-phosphate, glucose-6-phosphate, phytate, or mixture thereof.

9. The composition of claim 1, further comprising compound suitable for being immobilized on the support.

10. The composition of claim 9, wherein the compound comprises nucleic acid.

11. The composition of claim 10, wherein the nucleic acid comprises DNA, RNA, or mixture thereof.

12. The composition of claim 1, further comprising anionic or nonionic surfactant.

13. The composition of claim 12, wherein the anionic surfactant comprises sodium dodecyl sulfate.

14. The composition of claim 1, wherein the buffer comprises inorganic phosphate.

15. The composition of claim 14, wherein the inorganic phosphate comprises about 10 to about 200 mM sodium or potassium phosphate at pH of about 7 to about 10.

16. A composition comprising:
compound suitable for being immobilized on support; and
organic anion of formula: R(X)ₘ(Y)ₙ; in which:
R is alkyl, alkenyl, or alkynyl;
each X is independently carboxylate, phenol substituted with strongly electron withdrawing groups, phosphate, phosphonate, phosphinate, sulphate, sulphonate, thiocarboxylate, hydroxamate, or combination thereof;
each Y is independently amide, alcohol, ether, thiol, thioether, ester, thioester, borane, boric acid, or metal complex;
m is 1-7; and
n is greater than 1.

17. The composition of claim 16, wherein the compound comprises nucleic acid.

18. The composition of claim 17, wherein the nucleic acid comprises DNA, RNA, or mixture thereof.

19. The composition of claim 16, wherein the organic anion of formula I has the structure of represented by Formula III: 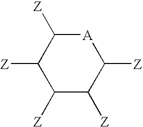
in which:
Z is —OH, —OPO₃⁻ or —CH₂—OPO₃⁻; and
at least one Z is —OPO₃⁻ or —CH₂—OPO₃⁻;
Y is —OH; and
A is —CH—OPO₃⁻ or O.

20. The composition of claim 19, wherein the organic anion is glucose-1-phosphate, glucose-6-phosphate, phytate, or mixture thereof.

21. The composition of claim 16, further comprising anionic surfactant.

22. A composition comprising:
a buffer effective for maintaining pH of aqueous composition at greater than or equal to about 6; and
neutral hydrophilic polymer of Formula V: 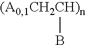
in which, A is absent, CH₂ or O;
n is about 100 to about 5000; and
B is —OH, —OC(O)CH₃, —CONH₂, —CONHR, —CONR₂, —OCH₃, —SH, —SCH₃, —COOR, —COSR, borane, boric acid, sulfone, amine oxide, or mixtures thereof;
the neutral hydrophilic polymer being effective to substantially decrease ring formation upon drying of a spot less than or equal to about 300 μm diameter on a support.

23. The composition of claim 22, wherein A is absent, n is about 600 to about 1300, and B is —OH.

24. The composition of claim 22, wherein the neutral hydrophilic polymer comprises polyvinyl alcohol.

25. The composition of claim 24, wherein the neutral hydrophilic polymer comprises and the polyvinyl alcohol comprises 88% hydrolyzed polyvinyl alcohol.

26. The composition of claim 24, wherein the neutral hydrophilic polymer comprises polyvinyl alcohol and the polyvinyl alcohol has a molecular weight of 31-51 kD.

27. The composition of claim 22, further comprising compound suitable for being immobilized on the support.

28. The composition of claim 27, wherein the compound comprises nucleic acid.

29. The composition of claim 28, wherein the nucleic acid comprises DNA, RNA, or mixture thereof.

30. The composition of claim 22, further comprising anionic or nonionic surfactant.

31. The composition of claim 30, wherein the anionic surfactant comprises sodium dodecyl sulfate.

32. The composition of claim 22, wherein the buffer comprises inorganic phosphate.

33. The composition of claim 32, wherein the inorganic phosphate comprises about 10 to about 200 mM sodium or potassium phosphate at pH of about 7 to about 10.

34. A composition comprising:
compound suitable for being immobilized on support; and
neutral hydrophilic polymer of Formula V: 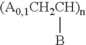
in which, A is absent, CH₂ or O;
n is about 100 to about 5000; and
B is —OH, —OC(O)CH₃, —CONH₂, —CONHR, —CONR₂, —OCH₃, —SH, —SCH₃, —COOR, —COSR, borane, boric acid, sulfone, amine oxide, or mixtures thereof;
the neutral hydrophilic polymer being effective to substantially decrease ring formation upon drying of a spot less than or equal to about 300 μm diameter on a support.

35. The composition of claim 34, wherein A is absent, n is about 600 to about 1300, and B is —OH.

36. The composition of claim 34, wherein the neutral hydrophilic polymer comprises polyvinyl alcohol.

37. The composition of claim 36, wherein the polyvinyl alcohol comprises 88% hydrolyzed polyvinyl alcohol.

38. The composition of claim 36, wherein the polyvinyl alcohol comprises polyvinyl alcohol with a molecular weight of 31-51 kD.

39. The composition of claim 34, wherein the compound comprises nucleic acid.

40. The composition of claim 39, wherein the nucleic acid comprises DNA, RNA, or mixture thereof.

41. The composition of claim 34, further comprising anionic surfactant.

42. A method of forming spots of a compound on a surface, the method comprising:
applying to the surface a composition comprising:
compound suitable for being immobilized on the surface; and
an organic anion of formula I: R(X)ₘ(Y)ₙ; in which:
R is alkyl, alkenyl, or alkynyl;
each X is independently carboxylate, phenol substituted with strongly electron withdrawing groups, phosphate, phosphonate, phosphinate, sulphate, sulphonate, thiocarboxylate, hydroxamate, or combination thereof;
each Y is independently amide, alcohol, ether, thiol, thioether, ester, thioester, borane, boric acid, metal complex;
m is 1-7; and
n is greater than 1; and
forming a spot on the surface.

43. The method of claim 42, wherein the organic anion is effective to substantially decrease ring formation upon drying of a spot less than or equal to about 300 μm diameter on a support.

44. The method of claim 42, wherein the composition further comprises a buffer effective for maintaining pH of aqueous composition at greater than or equal to about 7.5.

45. The method of claim 42, wherein applying comprises pin spotting or piezoelectric spotting.

46. A method of forming spots of a compound on a surface, the method comprising:
applying to the surface a composition comprising:
compound suitable for being immobilized on the surface; and
neutral hydrophilic polymer of Formula V: 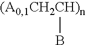
in which, A is absent, CH₂ or O;
n is about 100 to about 5000; and
B is —OH, —OC(O)CH₃, —CONH₂, —CONHR, —CONR₂, —OCH₃, —SH, —SCH₃, —COOR, —COSR, borane, boric acid, sulfone, amine oxide, or mixtures thereof; and
forming a spot on the surface.

47. The method of claim 46, wherein the organic anion is effective to substantially decrease ring formation upon drying of a spot less than or equal to about 300 μm diameter on a support.

48. The method of claim 46, wherein the composition further comprises a buffer effective for maintaining pH of aqueous composition at greater than or equal to about 7.5.

49. The method of claim 46, wherein applying comprises pin spotting or piezoelectric spotting.

50. An array of spots formed by the method of claim 42.

51. An array of spots formed by the method of claim 56.

52. A plurality of spots on a solid support, one or more of the spots comprising:
compound suitable for being immobilized on support; and
organic anion of formula: R(X)ₘ(Y)ₙ; in which:
R is alkyl, alkenyl, or alkynyl;
each X is independently carboxylate, phenol substituted with strongly electron withdrawing groups, phosphate, phosphonate, phosphinate, sulphate, sulphonate, thiocarboxylate, hydroxamate, or combination thereof;
each Y is independently amide, alcohol, ether, thiol, thioether, ester, thioester, borane, boric acid, metal complex;
m is 1-7; and
n is greater than 1.

53. A plurality of spots on a solid support, one or more of the spots comprising:
compound suitable for being immobilized on support; and
neutral hydrophilic polymer of Formula V: 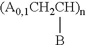
in which, A is absent, CH₂ or O;
n is about 100 to about 5000; and
B is —OH, —OC(O)CH₃, —CONH₂, —CONHR, —CONR₂, —OCH₃, —SH, —SCH₃, —COOR, —COSR, borane, boric acid, sulfone, amine oxide, or mixtures thereof.


SEQUENCE LISTING (9 sequences)

SEQ ID NO: 1 - DNA, 35 bp, Homo sapiens
cctgcgccagttgaatgccagtgagatagatamra

SEQ ID NO: 2 - DNA, 30 bp, Homo sapiens
gtctgagtcggagccagggcggccgccaac

SEQ ID NO: 3 - DNA, 30 bp, Homo sapiens
ctggttttctgctccttggtcctgggtgtc

SEQ ID NO: 4 - DNA, 30 bp, Homo sapiens
ctgtcccctttagagatcccacctgtcaga

SEQ ID NO: 5 - DNA, 30 bp, Homo sapiens
ttccttcatccctcttgtttcccaggtttt

SEQ ID NO: 6 - DNA, 35 bp, Homo sapiens
gccatgtgcagtctggttcaggttcataaatamra

SEQ ID NO: 7 - DNA, 30 bp, Homo sapiens
gccatgtgcagtctggttcaggttcataaa

SEQ ID NO: 8 - DNA, 30 bp, Homo sapiens
tgattgtgttctctgcctctggttgacctt

SEQ ID NO: 9 - DNA, 30 bp, Homo sapiens; identical to SEQ ID NO: 2